(19) United States
(12) Patent Application Publication
(10) Pub. No.: US 2026/0144825 A1
(43) Pub. Date: May 28, 2026

(54) METHODS OF TREATING EYE DISEASES USING OPTIC NERVE LAMINA REGION NEURAL PROGENITOR CELL-DERIVED SECRETED EXTRACELLULAR VESICLES AND FREE PROTEINS

(71) Applicant: University of Maryland, Baltimore, Baltimore, MD (US)

(72) Inventor: Steven Lance BERNSTEIN, Chevy Chase, MD (US)

(73) Assignee: University of Maryland, Baltimore, Baltimore, MD (US)

(21) Appl. No.: 19/119,547

(22) PCT Filed: Oct. 16, 2023
(86) PCT No.: PCT/US2023/035201

§ 371 Date: Apr. 9, 2025

Related U.S. Application Data
(60) Provisional application No. 63/416,099, filed on Oct. 14, 2022.

(51) Int. Cl. A61K 35/30; A61K 9/00; A61P 27/02

(52) U.S. Cl. CPC A61K 35/30; A61K 9/0048; A61P 27/02

(57) ABSTRACT

Soluble proteins and extracellular vesicles (EVs) produced by optic nerve lamina region neural progenitor cells (ONLR-NPCs) are provided. Such proteins and EVs include growth factors and survival factors that can be used in the treatment of optic nerve diseases, such as glaucoma. Also provided are methods of treating or preventing optic nerve diseases, such as glaucoma, using ONLR-NPC-based compositions.

### STATEMENT OF FEDERALLY SPONSORED RESEARCH AND DEVELOPMENT

This invention was made with government support under Grant Number EY032519 awarded by the National Institutes of Health. The government has certain rights in the invention.

### BACKGROUND

The retina is the light-sensitive neural tissue that detects and conveys visual information to the brain. Retinal ganglion cells (RGCs) serve as the channel through which input received by presynaptic partners in the retina is delivered to postsynaptic regions of the brain. RGCs initiate in the retina and have axons that emerge from the posterior of the eye to form the optic nerve (ON) which terminates in the brain. RGC axons are unmyelinated while in the retina, becoming myelinated as they pass through the anterior optic nerve lamina region and exit the eye [1].

The optic nerve lamina region (ONLR) is a transitional area containing a vascular plexus. It separates the unmyelinated RGC axons emerging from the eye from the myelinated axons in the distal optic nerve. The ONLR has a number of unusual characteristics. For example, it inhibits intraocular myelination, enables postnatal optic nerve myelination of growing axons, and modulates the fluid pressure differences between eye and brain. Moreover, it has recently been shown that the ONLR contains a niche of adult neural progenitor cells (NPCs) capable of generating all macroglial cell types for the anterior optic nerve. Early ONLR-NPC loss causes regional dysfunction and myelination defects. Age-related depletion may help explain development of ON diseases such as open angle glaucoma [8].

ONLR defects and damage are associated with the majority of age-associated intrinsic optic nerve diseases, including nonarteritic anterior ischemic optic neuropathy (NAION) and primary open angle glaucoma (POAG). The physiological basis for the development of these diseases remains unclear.

NAION is currently untreatable, and believed to be initiated by a single vascular insult centered at the ONLR [2]. While NAION is associated with a compartment syndrome that damages RGC axons and causes rapid RGC loss, there is variability in the ability of individuals to recover. ˜50% of individuals do not improve, ˜30% of individuals improve somewhat, and ˜20% of individuals experience further visual loss [3]. The chances of improvement are associated with decreased age [4], with a strong trend toward improvement even in studies that did not show statistically significant association [3], suggesting that additional, unexplained factors contribute to both long-term recovery and degeneration.

POAG is associated with increased intraocular pressure (IOP) [5], but is also now believed to be largely a neurodegenerative disease [6], with IOP being only one factor in the development and progression of the condition [5, 7]. Low tension forms of glaucoma (so-called normal tension glaucoma) are not associated with elevated IOP, but reducing IOP is the only currently accepted treatment.

If effective treatments are to be developed for these untreatable diseases, it is crucial to identify the variables that contribute to disease susceptibility, the causes of disease progression, and the factors enhancing repair, to create more effective treatments. The present disclosure is directed to such needs and other important goals.

### BRIEF SUMMARY

The present inventors recently discovered that primate and rodent ONLRs contain a neural stem cell/neural progenitor cell (NSC/NPC) niche in the anterior ON that declines during aging [8]. This discovery is a paradigm shift for understanding and designing future treatments for ON disease. It fills critical gaps in our understanding of 1) how normal ON function is maintained and renewed in humans; and 2) RGC resistance factors to stressors. The age-associated decline in ONLR NPCs provides a novel explanation for why intrinsic ON diseases such as POAG develop later in life. Enhancing ONLR-NPC functions will enable development of new ON disease treatments and may greatly improve outcomes. Experiments discussed herein demonstrate that ONLR-NPC secrete factors such as soluble proteins and extracellular vesicles (EVs) (membrane coated vesicles containing both growth factors and RNAs) that are responsible for maintaining RGC resistance to stress, and indicate that administration of these factors improves ON disease outcomes.

The present invention generally provides methods for using proteins derived from optic nerve lamina region neural progenitor cells (ONLR-NPCs) in the treatment and/or prevention of optic nerve diseases, in enhancing RGC survival, and in enhancing RGC neurite outgrowth.

In addition, the present invention generally provides methods for using EVs derived from ONLR-NPCs in the treatment and/or prevention of optic nerve diseases, in enhancing RGC survival, and in enhancing RGC neurite outgrowth.

Similarly, the present invention generally provides methods for using factors isolated from EVs derived from ONLR-NPCs in the treatment and/or prevention of optic nerve diseases, in enhancing RGC survival, and in enhancing RGC neurite outgrowth.

Furthermore, the present invention generally provides methods for using factors identified as present in the EVs derived from ONLR-NPCs in the treatment and/or prevention of optic nerve diseases, in enhancing RGC survival, and in enhancing RGC neurite outgrowth.

Soluble proteins and EVs produced by and/or isolated from ONLR-NPCs, as defined herein, form the basis of the present invention. The invention thus includes, but is not limited to, factors such as (i) ONLR-NPC-secreted soluble proteins, and compositions comprising these proteins, (ii) ONLR-NPC-produced EVs, (iii) ONLR-NPC conditioned media containing ONLR-NPC-secreted soluble proteins and/or ONLR-NPC-produced EVs, (iv) ONLR-NPC EV lysates, (v) ONLR-NPC EV extracts, and (vi) any combinations of factors (i)-(v). In addition, various factors secreted by ONLR-NPCs may be recombined in multiple combinations using nanoparticle or liposome-type delivery systems.

The invention includes methods of treating or preventing optic nerve diseases, such as glaucoma, using the factors of one or more of (i)-(vi). The invention also includes methods of enhancing retinal ganglion cell (RGC) survival using factors of one or more of (i)-(vi). The invention further includes methods of enhancing RGC neurite outgrowth using factors of one or more of (i)-(vi).

In particular, and in a first embodiment, the invention is directed to soluble proteins secreted by optic nerve lamina region neural progenitor cells (ONLR-NPCs), and compositions comprising these proteins. The ONLR-NPCs from which the secreted proteins are obtained in this and the other embodiments and aspects of the invention are characterized, for example, as nestin(+), SOX2(+), GFAP (+), HOPX(+), NG2(−) cells.

In a second embodiment, the invention is directed to ONLR-NPC EVs. The ONLR-NPCs from which the EVs are obtained in this and the other embodiments and aspects of the invention are characterized, for example, as nestin(+), SOX2(+), GFAP (+), HOPX(+), NG2(−) cells. The invention includes single ONLR-NPC EVs as well as populations of ONLR-NPC EVs.

In a third embodiment, the invention is directed to ONLR-NPC conditioned media containing ONLR-NPC-secreted soluble proteins and/or ONLR-NPC-produced EVs, obtained from a culture of ONLR-NPCs, such as an in vitro cell culture of ONLR-NPCs that have been cultured under conditions promoting secretion of soluble proteins and release of EVs into the cell culture media. The ONLR-NPC conditioned media may be prepared by growing the ONLR-NPCs for one or more days in a defined tissue culture medium, optionally containing Fibroblast Growth Factors-2 and -4 and Matrigel coating at concentrations of 10-15%. The ONLR-NPC conditioned media may be collected by decanting the sterile medium, followed by concentration and dialysis or gel filtration to eliminate both uninvolved medium, ions and large molecular weight compounds, such as bovine serum albumen, while retaining the proteins and EVs.

In a fourth embodiment, the invention is directed to an ONLR-NPC EV lysate. ONLR-NPC EV lysates comprise the material collected from ONLR-NPC EVs that are broken apart by mechanical, chemical or physical means. ONLR-NPC EV lysates may be prepared by collecting the EVs from ONLR-NPC culture medium, or obtained directly from ONLR-NPCs by lysing the cells or other suitable means, followed by separation via centrifugation and washing, and then lysis of the isolated EVs via mechanical, chemical or physical means to produce an EV lysate.

In a fifth embodiment, the invention is directed to ONLR-NPC EV extracts. These EV extracts may be a collection of selected factors isolated from EVs, or a collection of one or more selected factors known to be present in ONLR-NPC EVs but produced by means other than the ONLR-NPCs and combined to form an “artificial” ONLR-NPC extract, and combinations thereof. ONLR-NPC extracts may thus be prepared by direct isolation of selected factors from the ONLR-NPC EVs via lysis and recovery, as well as by combining selected individual factors into an “artificial” ONLR-NPC EV extract. An exemplary artificial ONLR-NPC EV extract is an artificial extract comprising each of Fibroblast Growth Factor 2 (FGF-2); Ciliary Neurotrophic Factor (CNTF); Transforming Growth Factor-Beta 2 (TGF-β2); one or more of Angiotensinogen, an Angiotensinogen peptide fragment, and Angiotensin; Alpha-2 macroglobulin; Thrombospondin-1; Ceruloplasmin; and Pigment Epithelium-derived Factor (PEDF). The “lysates” of the invention will include all of the factors that are recovered from a lysed EV, while an “extract” will include only those factors preselected to be present in the extract. Relevant factors include, but are not limited to, proteins, such as growth factors, and nucleic acids, such as miRNA. Additionally, exosomal-identified factors, e.g. the ONLR-NPC EV extracts of the invention, may be encapsulated into liposomes or specifically formulated nanoparticles to create an artificial EV-like environment containing the selected ingredients in an artificial EV.

In a sixth embodiment, the invention is directed to methods of (a) treating or preventing an optic nerve disease, (b) enhancing retinal ganglion cell (RGC) survival, and/or (c) enhancing RGC neurite outgrowth. In one aspect, each of these methods comprise administering to a subject in need thereof a therapeutically effective amount of a composition comprising one or more of:
(i) ONLR-NPC-secreted soluble proteins,
(ii) a population of ONLR-NPC-produced EVs,
(iii) ONLR-NPC conditioned media containing ONLR-NPC-secreted soluble proteins and/or ONLR-NPC-produced EVs,
(iv) ONLR-NPC EV lysates, and
(v) ONLR-NPC EV extracts,
   thereby (a) treating or preventing an optic nerve disease, (b) enhancing retinal ganglion cell (RGC) survival, and/or (c) enhancing RGC neurite outgrowth. The ONLR-NPC EV extract of (v) may be in the form of an artificial EV.

In one aspect, the optic nerve disease is open-angle glaucoma, such as primary open-angle glaucoma (POAG). In another aspect, the optic nerve disease is angle-closure glaucoma. In a further aspect, the optic nerve disease is optic nerve hypoplasia, optic nerve hypomyelination, regional axonal dysfunction, nonarteritic anterior ischemic optic neuropathy (NAION), or optic neuritis. In one aspect, the administering is administration to the eye of the subject via means that include, but are not limited to, topical application (e.g. eye drops), subconjunctival injection, intravitreal injection, and retrobulbar injection.

In one aspect, the composition administered to the subject is a pharmaceutical formulation comprising a pharmaceutically acceptable carrier and one or more of:
(i) ONLR-NPC-secreted soluble proteins,
(ii) a population of ONLR-NPC-produced EVs,
(iii) ONLR-NPC conditioned media containing ONLR-NPC-secreted soluble proteins and/or ONLR-NPC-produced EVs,
(iv) ONLR-NPC EV lysates, and
(v) ONLR-NPC EV extracts.
   The ONLR-NPC EV extracts of (v) may be in the form of an artificial EV.

As indicated above, the ONLR-NPCs of each embodiment and aspect of the invention are characterized based on the expression or lack of expression of certain markers by the cells. For example, in some aspects and embodiments, the ONLR-NPCs are characterized as nestin(+), SOX2(+), GFAP (+), HOPX(+), NG2(−) cells. In a related aspect, the ONLR-NPCs are characterized as nestin (+), SOX2(+), GFAP(+), HOPX(+), NG2(−), vimentin(+), BDNF(+) cells.

In some aspects, the ONLR-NPCs are further characterized based on their ability to secrete one or more growth factors. These growth factors include one or more of Ciliary Neurotrophic Factor (CNTF), Fibroblast Growth Factor 1 (FGF-1), Fibroblast Growth Factor 2 (FGF-2), Insulin-like Growth Factor 2 (IGF-2), Midkine (Neurite Growth-promoting Factor 2), Platelet-derived Growth Factor alpha (PDGFA), Platelet-derived Growth Factor C (PDGFC), Transforming Growth Factor-Beta 1 (TGF-β1), Transforming Growth Factor-Beta 2 (TGF-β2), and Vascular Endothelial Growth Factor A (VEGFA). In a related aspect, the ONLR-NPCs secrete at least five of the growth factors listed in Table 1 (i.e. CNTF, FGF-1, FGF-2, IGF-2, Midkine, PDGFA, PDGFC, TGF-β1, TGF-β2 and VEGFA). Alternatively or in addition, the ONLR-NPCs express one or more of the additional proteins listed in Table 2. In a further related aspect, the ONLR-NPCs secrete each of the growth factors listed in Table 1, or express each of the proteins listed in Table 2, or each of the growth factors and proteins listed in Tables 1 and 2.

In relevant aspects of the invention, compositions and formulations comprising ONLR-NPC-secreted soluble proteins comprise one or more of CNTF, FGF-1, FGF-2, IGF-2, Midkine, PDGFA, PDGFC, TGF-β1, TGF-β2 and VEGFA. In a related aspect, compositions and formulations comprising ONLR-NPC-secreted soluble proteins comprise at least five of the growth factors listed in Table 1 (i.e. CNTF, FGF-1, FGF-2, IGF-2, Midkine, PDGFA, PDGFC, TGF-β1, TGF-β2 and VEGFA).

In relevant aspects of the invention, ONLR-NPC-secreted soluble proteins contained in ONLR-NPC conditioned media are one or more of CNTF, FGF-1, FGF-2, IGF-2, Midkine, PDGFA, PDGFC, TGF-β1, TGF-β2 and VEGFA. In a related aspect, the secreted soluble proteins are at least five of the growth factors listed in Table 1 (i.e. CNTF, FGF-1, FGF-2, IGF-2, Midkine, PDGFA, PDGFC, TGF-β1, TGF-β2 and VEGFA).

In relevant aspects of the invention, the ONLR-NPC EV extract comprises one or more of Alpha-2 macroglobulin, Angiotensinogen, an Angiotensinogen peptide fragment, Angiotensin, Antithrombin 3-Serpin C1, CD9, CD81, Ceruloplasmin, Insulin-like Growth Factor-2 (IGF-2), LGI4, Pigment Epithelium-derived Factor (PEDF), Osteogenic growth factor 1 (OSTF1), Thrombospondin-1, and Adipocyte Enhancing Binding Protein-1 (AEBP1) (Table 3).

In a related aspect, the ONLR-NPC EV extract comprises at least five of Alpha-2 macroglobulin, Angiotensinogen, an Angiotensinogen peptide fragment, Angiotensin, Antithrombin 3-Serpin C1, CD9, CD81, Ceruloplasmin, Insulin-like Growth Factor-1 (IGF-1), LGI4, Pigment Epithelium-derived Factor (PEDF), Osteogenic growth factor 1 (OSTF1), Thrombospondin-1, and Adipocyte Enhancing Binding Protein-1 (AEBP1).

In a related aspect, the ONLR-NPC EV extract comprises each of Alpha-2 macroglobulin, Antithrombin 3-Serpin C1, CD9, CD81, Ceruloplasmin, Insulin-like Growth Factor-1 (IGF-1), LGI4, Pigment Epithelium-derived Factor (PEDF), Osteogenic growth factor 1 (OSTF1), Thrombospondin-1, Adipocyte Enhancing Binding Protein-1 (AEBP1), and one or more of Angiotensinogen, an Angiotensinogen peptide fragment, and Angiotensin.

Alternatively or in addition, the ONLR-NPC EV extract comprises one or more of the additional proteins listed in Table 4. In a further related aspect, the ONLR-NPC EV extract comprises each of the growth factors listed in Table 3, or each of the proteins listed in Table 4, or each of the growth factors and proteins listed in Tables 3 and 4.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1. A. Hematoxylin and Eosin (H&E) stained section of the primate ONLR. The lamina/ONLR is at the junction of the eye and optic nerve, with connective tissue columns and surrounded by the sclera and optic nerve sheath. B. Immunohistochemical staining of the ONLR contains the unmyelinated RGC axons (SMI312(+); in red), while the myelinated portion of the ON (MBP; in green) begins directly below. C. ONLR vascular supply. The ONLR (‘Lamina’ in this drawing) has a vascular plexus within it, receiving blood from the retina, optic nerve and choroid.

FIG. 2. Characteristics of the ONLR-NPC niche (rodent). A. The ONLR lies between the retina and the distal ON. B. ONLR vessels form a plexus with inputs from retina, optic nerve and surrounding choroidal circulation. C. Like other neural stem niches, the ONLR has reduced relative levels of aquaporin-4 (AQP4), compared with the retina and remaining ON; this is a characteristic of other neural progenitor niches [9]. D. ONLR-NPCs express nestin, as seen in this transgenic mouse which expresses GFP under the nestin promotor.

FIG. 3. ONLR-NPC characteristics in rodents and humans. A. Nestin(+)/SOX2(+) NPCs form a niche in the ONLR. Immunohistochemical analysis: SOX2(+) nuclei are shown in green, while nestin is red. Inset: there are many fewer SOX2(+) cells in the distal ON, and almost a complete absence of fibrillar nestin. B. Neurospheres generated from mouse ONLR-NPCs. Upper inset: The ONLR generates many more neurospheres (blue bars) than does the distal ON (red bars), and this ability declines from pup (20 d/o) to adult (60 d/o). C. Immunohistochemical analysis of glial cell generation from ONLR-NPC cultures. Both astrocytes (GFAP(+), in green) and oligodendrocytes (O₄(+), in red) are generated after fetal bovine serum is added and growth factors are withdrawn. D. ONLR-NPCs can generate Tuj1(+) cells (in red) consistent with neurons. E. Graph: ONLR-NPCs decline during aging. Mouse ONLR and distal ONs were quantified for SOX2(+) cells. SOX2(+) cells declined from 1 mo-2 yr in the ONLR (black bars), while SOX2(+) cells in the distal ON remained relatively stable (white bars). F. SOX2(+)/Nestin(+) ONLR-NPC niche in human ONLR. Tissue taken from an 8 mo preterm fetus. There is considerable concentration of nestin immunoreactivity (in red) in the ONLR, as well as SOX2(+) nuclei (in green), compared with the rest of the ON.

FIG. 4. Loss of SOX2(+)/nestin(+) ONLR-NPCs in SOX2-Cre-ERT2×ROSA-LoxP diphtheria toxin antigen (DTA) double heterozygous mice two weeks following retrobulbar 4-hydroxytamoxifen (4OHT) administration. The 4OHT binds to the ERT2 receptor and activates Cre, leading to expression of diphtheria toxin (DTA) in the SOX2(+) cells of double transgenic mice, leading to their elimination. All panels are from the same animal. A, C: vehicle-treated nerve. B, D: 4OHT-treated nerve. There is a selective loss of ONLR SOX2 (+) nuclear numbers and nestin immunoreactivity signal on the 4OHT treated side, while the vehicle treated side shows normal signal levels. The inset shows the quantification of SOX2(+) nuclei on each side (±sd; n=3 sections/nerve).

FIG. 5. Expression of stem cell markers in human ONLR-NPCs, neurosphere production and age-related changes. A. Immunohistochemical analysis of Nestin/SOX2 expression in a young (22 y/o) ONLR. The NPCs are grouped in columns within the region. Sox2(+) nuclei are green; Nestin expression is red. B. Depletion of NPCs in the elderly ONLR. There are no SOX2(+)/nestin(+) cells remaining in this 70 y/o donor. C. Age-related decline in ONLR-nestin expression (densitometry). There is a steep decline after 40 years of age. D. Neurospheres derived from human ONLR-NPCs in culture. No neurosphere formation is generated from cells in the distal ON.

FIG. 6. Structural and functional analyses of human ONLR-NPC exosomes. A. Ultrastructural analysis. ONLR-exosomes were collected by ultracentrifugation, shadowed using uranyl acetate, and imaged via transmission electron microscopy (TEM). Exosomes ranged from 30-120 um in diameter. B. ONLR-NPC-derived exosomes enhance RGC survival and enhance RGC-neurite extension in culture. ONLR-NPC exosomes from conditioned medium enhanced rat RGC survival by 225±10%, compared with exosomes from fibroblasts, and also enhanced the number of RGCs extending neurites. C. ONLR-NPC-derived exosomes enhance RGC neurite length by >8-fold, compared with fibroblast exosomes. D. Immuno-histological comparison of BIII-tubulin (a neuron-specific marker) in neurite extension of RGC cultures treated with exosomes from ONLR-NPCs and fibroblasts. There is a robust extension of RGC neurites treated with ONLR-NPC exosomes, compared with fibroblast-derived exosomes.

FIG. 7. Measurement of intraocular pressure after microbead induced glaucoma in Sprague-Dawley rats. Intact (no microbead) eyes show normal pressure. Microbead-treated eyes used for either fibroblast exosomes or ONH exosomes show elevated pressure (ocular hypertension), and this pressure elevation continues for over 40 days.

FIG. 8. Measurement of retinal ganglion cell survival after intravitreal (intraocular) injection of human ONLR-NPC exosomes in a rat model of glaucoma. BMSC: Bone marrow derived stem cell (exosome) supplementation. hONH: Human ONH/ONLR-NPC exosome supplementation. rONH: rat ONLR-NPC exosome supplementation.

FIG. 9. 3 day retinal cultures treated with EVs isolated from either primary rat skin fibroblast or ONLR-NPC cultures. RGC nuclei were stained green (Brn3a(+)), while Tuj1(+) neurites were stained red. The combination of red and green gives a yellow color. RGCs are indicated in both panels by arrows. The quantification of RGC neurite lengths are shown in the ONLR-NPC EV treated panel. Scale bars are shown at the bottom in each panel. The fibroblast EV treated retinal cells did not extend noticeable neurites, compared with the ONLR-NPC EV treated retinal cells.

FIG. 10. Angiotensin (AgT) is expressed exclusively in the ONH and secreted in EVs by ONLR-NPCs, and the RGC-angiotensin receptor is strongly upregulated in RGCs after ONC. Similarly, ceruloplasmin (Cp) is identified in the ONH exclusively expressed in ONLR-NPCs and plays a major role in iron metabolism in neurons, where it can inhibit oxidative damage associated with ferritin. Transferrin (Tf) also plays an important role in RGC function, it is strongly expressed in a limited number of cell types, including ONLR-NPCs and oligodendrocytes. Ceruloplasmin is known to play a major role in iron metabolism. Ceruloplasmin therefore plays an important role in in RGC function after stress, as shown by the fact that the genes for the iron storage protein ferritin (both ft11 and fh11) are strongly upregulated in RGCs after ONC, suggesting that at least ONLR-NPC-EV secreted Cp that is taken up by the RGC axons may play a role in regulating RGC stress-related iron homeostasis. Alpha-2 Macroglobulin (A2M) is expressed exclusively in the ONH by ONLR-NPCs, and A2M plays an important role in APP metabolism.

FIG. 11. Rat ONLR-NPC EVs stimulate RGC survival, Neurite extension and outgrowth. Comparison between Fibroblast- and ONLR-NPC derived EVs (3 wells/condition). A. RGC survival. ONLR-NPC EVs enhanced RGC survival >2 fold. B. Neurite extension. ONLR-NPC EVs stimulated 7-fold greater neurites than Fibroblast EVs. C. Mean Neurite length. ONLR-NPC EVs stimulated neurite extension >4 fold than did EVs from Fibroblast cultures.

FIG. 12. Cell groups identified by rat optic nerve head (ONH) single cell sequencing (sc-seq). The group of ONLR-NPCs are indicated by an arrow in A. Expression levels in different cell groups are shown by increasing amount of red color in remaining (B-I) panels. Extracellular vesicle secreted proteins include alpha-2 macroglobulin (B.), ceruloplasmin (C.), angiotensinogen (D.), thrombospondin (E.), and PEDF (F.). Single cell sequencing shows that the ONLR-NPCs express (and then secrete) CNTF (G.), FGF2 (H.) and TGFb2 (I.) growth factors at high levels.

FIG. 13. Shown in panels A-G is confirmation that identified growth factors are required during RGC stress (optic nerve crush), as shown by the changes in the expression levels of their specific growth factor receptors in RGCs in naïve cells, and after optic nerve crush. The expression levels were obtained by single cell sequencing of retinal ganglion cells (RGCs) in naïve and post-optic nerve crush as reported by Tran [66].

### DETAILED DESCRIPTION

### I. Definitions

Unless otherwise noted, technical terms are used according to conventional usage. Definitions of common terms in molecular biology may be found, for example, in Benjamin Lewin, Genes XII, published by Jones & Bartlett Learning, 2014 (ISBN 1449659853); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

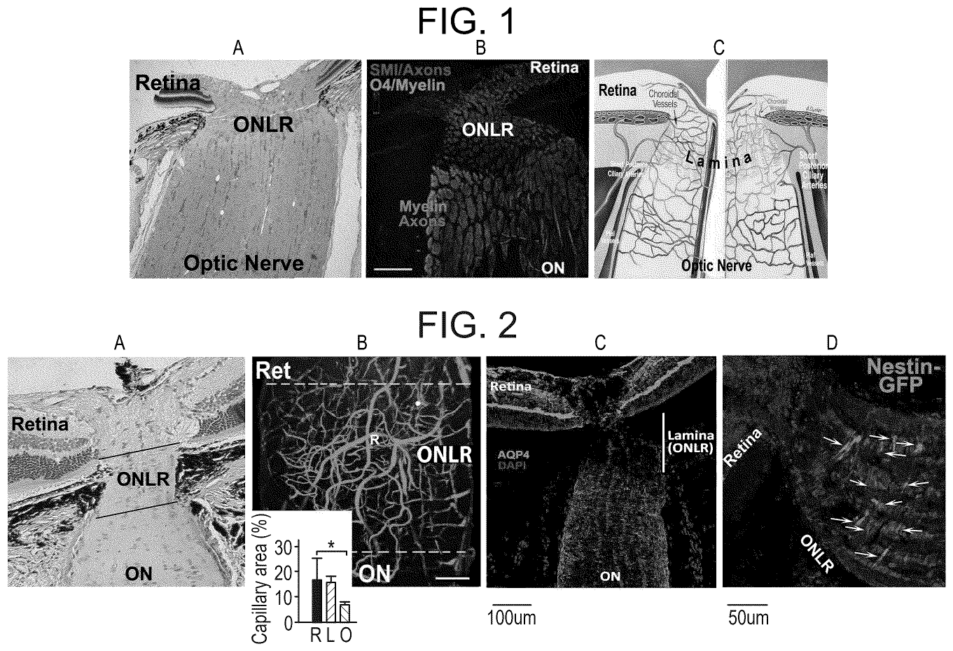

The optic nerve laminar region (ONLR) is the unmyelinated region in the most anterior portion of the optic nerve (ON) (FIG. 1A, B; FIG. 2A), and encloses the retinal ganglion cell (RGC) axons as they emerge from the eye. In primates and rodents, the ONLR possesses a unique vasculature plexus having contributions from three different circulatory sources: the retina, underlying choriocapillaris, and intrinsic optic nerve vasculature (FIG. 1C; FIG. 2B) [10-12]. This complex vasculature enables the ONLR to sample the metabolism of the contributing components. This is similar to other CNS stem cell niches, which are responsive to their surround [9]. Rat and mouse ONLR have similar composition but with simplified contributions. For example, collagen columns in primate ONLR are replaced by collagen plates in the rat, and are minimal in the mouse; the amount of retinal and choroidal vascular contribution is reduced, but still present in the rat and mouse.

Initial characterization of the ONLR suggested neural progenitor cells (NPCs) might be present in the region. For example, glial cells in the mouse ONLR were found having peculiar properties that span the individual axonal bundles passing from eye to ON [13], but were not identified as NPCs. ONLR ‘astrocytes’ (GFAP (+) cells) were found that have a phagocytic ability [14], similar to that shown by NPCs [15]. The ONLR also possesses a restrictive property that blocks progressive retinal myelination from the ON [16], although CNS neurons themselves are permissive for myelination throughout life [53]. These unusual ONLR properties were not previously assumed to be associated with NPCs. ON-reparative cells were previously assumed exclusively to be derived from NG2(+) antigen expressing precursor cells (oligodendrocyte precursor cells: OPCs) that form a scattered cell pool within the nerve [54-56]. NPCs previously identified in the adult eye have been localized in the retina and ciliary body [57], including a subpopulation of glia resembling Mueller cells [17-18]. These cells have the ability to differentiate into retinal photoreceptor neurons and potentially other outer retinal neuron types. A subset of retinal pigment epithelial (RPE) cells can give rise to mesenchymal cell types, and are considered mesenchymal stem cells [19]. Induced pluripotent cells (iPS) can generate eye structures and nearly complete retinae [58], but not optic nerves.

The previous investigations into ONLR-NPCs, their regenerative abilities and a detailed cellular analysis was limited in part because the ONLR represents only a miniscule region in the anterior ON, the difficulty in isolating tissue from this intermediate position between eye and ON, and a high concentration of connective tissue complicating intrinsic cellular isolation and cell culture techniques.

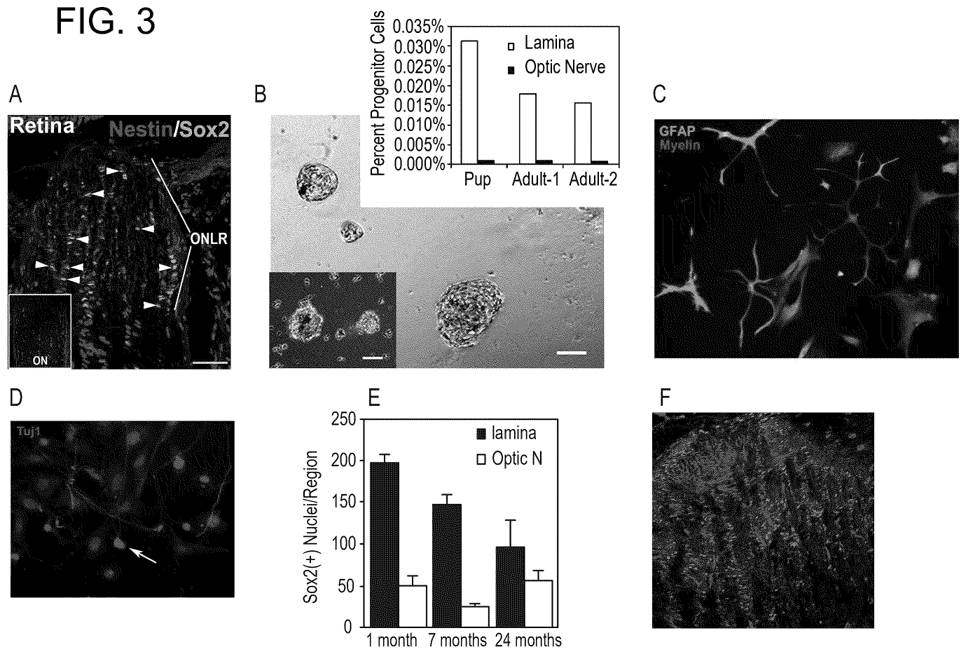

These problems were solved by the present inventors. Previous ONLR cell cultures were generated from primary explants, using techniques that typically isolate astrocytes and fibroblast-like cells, rather than cells with primordial characteristics [59-61]. These cells were cultivated for extended periods in conditions that caused differentiation, such as cultivation with fetal bovine serum. It is therefore not surprising that NPCs were overlooked. Additionally, it was assumed that the entire adult ON replaces oligodendrocytes exclusively from scattered resident NG2 antigen (+) oligodendrocyte precursor cells (OPCs), representing a prenatally generated, self-replicating cellular population [62]. OPCs can migrate and differentiate into both type 1 astrocytes and oligodendrocytes [63]. However, NPCs can not only self-replicate, but can give rise to daughter cells that differentiate into neurons, type 1 and type 2 astrocytes, and oligodendrocytes (FIG. 3C).

NPC identity is partially defined by the co-expression of SOX2, nestin, and GFAP proteins. Adult NPCs also form neurospheres in low adhesion condition, the cells can differentiate into all neural forms, and the cells are depleted during aging. As shown herein, the ONLR contains a population of SOX2(+)/Nestin(+)/GFAP(+) cells (FIG. 3A). ONLR-NPCs also express HOPX protein. These ONLR NPCs can form neurospheres in low adhesion medium (FIG. 3B), differentiate into both glia and neurons (FIG. 3C, D), and decline during the aging process (FIG. 3E). They thus fulfill all requirements for NPC characteristics. The ONLR-NPC niche is also found, greatly expanded, in humans (FIG. 3F).

Optic nerve regeneration and repair previously has been considered to involve only remyelination [64], rather than neuronal replacement from a stem/neural progenitor cell niche. This approach does not enable consideration of repair of primary defects involved in OAG, nor can it be useful to repair degenerative ON diseases such as NAION. There has been no understanding of why laminar defects predispose to open angle glaucoma (OAG). The ONLR was not previously believed to contain an NPC niche. However, the ONLR's regional vascular complexity, its role in contributing to unidirectional myelination, and its distinctive lack of the water channel aquaporin 4 (AQP4) compared with either retina or optic nerve (see FIG. 2C), which is typically found at high concentrations in neural tissue, were all suggestive of a neural progenitor cell niche [9]. NPCs derived from an ONLR niche would be ideally placed to contribute large numbers of cells for the postnatal growing ON, to replace age-associated glial cell loss, and to enhance repair following damage from blinding ON-related diseases such as open angle glaucoma (OAG) and NAION.

Identification of an ONLR-NPC niche (by the present inventors), and replacement of these cells, is key to a new approach with an enhanced ability to prevent or repair glaucoma-related damage, and repair other age-related anterior optic nerve degenerations.

The present inventors discovered that primate and rodent ONLRs contain a neural stem cell/neural progenitor cell (NSC/NPC) niche in the anterior ON that declines during aging [8]. This discovery is a paradigm shift for understanding and designing future treatments for ON disease. It fills critical gaps in our understanding of 1) how normal ON function is maintained and renewed in humans; and 2) RGC resistance factors to stressors. The age-associated decline in ONLR NPCs provides a novel explanation for why intrinsic ON diseases such as POAG develop later in life. Thus, ONLR-NPCs exist to support early ON growth; in adults, these cells also suppress RGC stress. Enhancing ONLR-NPC functions will enable development of new ON disease treatments, and may greatly improve outcomes. The experiments discussed herein will tests the hypothesis that ONLR-NPC secrete specific factors working through at least two complementary systems (soluble protein factors and vesicle-bound factors) that play a role in regulating the ONH environment and RGC stress. Factors secreted by extracellular vesicles s (membrane coated vesicles containing both growth factors and RNAs) are responsible for maintaining RGC resistance to stress and administration of these factors will improve ON disease outcomes.

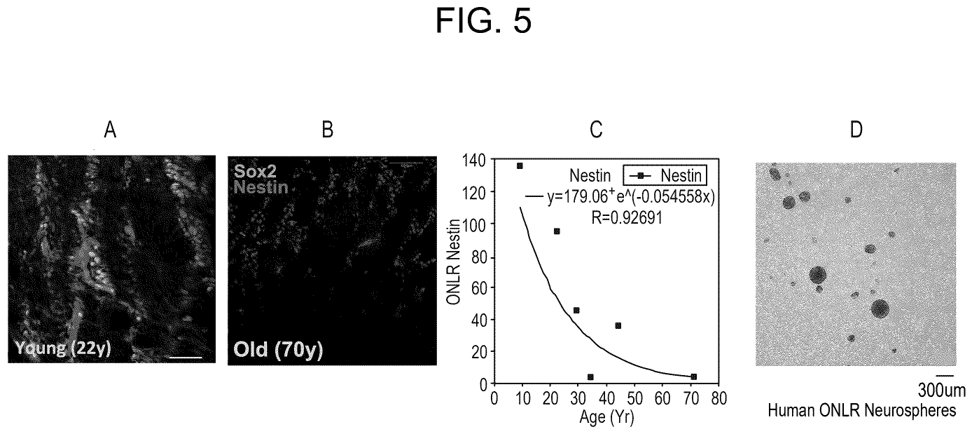

Data in support of the hypothesis includes previous work that demonstrated neural stem/progenitor cells are found in the ONLR. Adult neural stem/progenitor cells (aNSC/NPCs) maintain regenerative functions within the adult animal [20, 21], and can differentiate into both glial and neuronal cell forms [22]. aNSC/NPCs consist of numerous subtypes with definite preferences for different adult populations [23]. aNPCs and aNSCs decline during aging [24]. These cells are typically found in stem cell ‘niches’ defined by the presence of a vascular plexus (FIG. 2B), reduced levels of aquaporin 4 relative with their surround (FIG. 2C). There is also expression of stem cell protein markers such as nestin (FIG. 2D (green) and FIG. 5A (red)).

Oligodendrocyte progenitor cells (OPCs) are more differentiated than NPCs, largely involved in oligodendrocyte production, and widely distributed in the optic nerve. The postnatal ON was previously believed to only contain OPCs, in addition to astrocytes and oligodendrocytes [25]. OPCs also express the stem marker SOX2, and NG2/CSPG and PDGFRa [26], while NSC/NPCs express both SOX2 and nestin, but not NG2 or PDGFRa [27,28]. aNSCs and aNPCs can form neurospheres in low adherence medium (which OPCs cannot). NSC/NPCs can give rise to all neural forms (neurons, OPCs, oligodendrocytes, different astrocyte forms). It was recently discovered that the ONLR contains a previously undocumented NSC/NPC niche [8], that the progenitors in this niche decline during aging and when isolated form neurospheres in low adhesion culture conditions (FIG. 5A-D).

It is now generally recognized that NSC/NPCs are responsible for maintaining adult CNS tissue health [29,30]. Their loss can result in aging of CNS tissue [31]. NPCs secrete various growth factors, including IGF-1, VEGF and BDNF [32], which support surrounding neurons and enable repair. They engage in immune cross-talk with surrounding inflammatory cells [33,34]. An NPC niche at the precise site of RGC-axon emergence into the nerve is a critical and previously unsuspected component in RGC survival and optic nerve integrity in ocular stress and ON diseases. ONLR-NPC loss may increase RGC susceptibility to damage from stress.

As indicated above, ONLR-NPCs secrete membrane coated vesicles, termed extracellular vesicles (EVs), that generally range in size from 30-190 nm. A particular form of EVs are exosomes which generally range in size from 30-130 nm. Because of similarities in structure and function, all references herein to “EVs” should be understood to include “exosomes” unless explicitly stated otherwise.

When secreted by cells, EVs can transfer growth factors and proteins (such as insulin), mRNAs and other communication-related molecules such as microRNAs (miRNAs) from cell to cell. miRNAs suppress specific gene activities; proteins provide specific activities. NSC/NPCs can actually induce youthful neural phenotypes by EV secretion into the CNS cerebral spinal fluid in elderly mice [35], enabling recovery of hypothalamic function. Mesenchymal stem cell (MSC) derived EVs can improve neurogenesis and neural function after CNS stroke [36,37], and can induce a neuroprotective inflammatory state after injury [38,39]. MSC-derived EVs have been shown to rescue RGCs in culture and also in glaucoma models [40]. Exosomal compositions from both MSCs and NSCs can change and improve their rescue ability for RGCs and CNS ischemia according to the required response by the environment [41,42]. If EVs from non-neural derived MSCs are neuroprotective, and if NSC-derived EVs have such powerful neuroregenerative responses on hippocampal neurons, the effect of ONLR-NPC-secreted EVs may be harnessed in the treatment of eye diseases and improving RGC survival in stressful situations.

Work by the present inventors has confirmed the presence of an NPC niche in the ONLR [8]. Like other neural stem niches, the ONLR has reduced relative levels of aquaporin-4 (AQP4) (FIG. 2C). ONLR-NPCs express nestin, as seen in a transgenic mouse which expresses GFP under the nestin promotor (FIG. 2D).

As described herein, this population of ONLR-NPCs possess properties typically found in NPCs. For example, ONLR-NPCs express SOX2, as well as a number of NPC-specific proteins including nestin, Glial Fibrillary Acidic Protein (GFAP) and vimentin. ONLR-NPCs also express NPC-associated growth factors such as Transforming growth factor Beta-I (TGFβ1). Thus, the postnatal ONLR-NPC niche is capable of generating both types of glial cells in the ON, and enabling normal postnatal ON growth, myelination, fluid regulation and cellular replacement. ONLR-NPCs do not express Neural/Glial Antigen 2 (NG2).

### Optic Nerve Lamina Region Neural Progenitor Cells (ONLR-NPCs)

The present invention is directed, in part, to soluble proteins and EVs produced by and obtained from optic nerve lamina region neural progenitor cells (ONLR-NPCs). ONLR-NPCs are characterized as cells located in the ONLR of humans and rodents, and further defined in the paragraphs below.

The ONLR-NPCs can be defined based on the expression, or lack thereof, of selected marker proteins. The ONLR-NPCs can thus be defined as nestin(+), SOX2(+), GFAP(+), HOPX(+), NG2(−) neural cells. SOX2 is the transcription factor SRY (sex determining region Y)-box 2. GFAP is glial fibrillary acidic protein. HOPX is the homeodomain-only protein. NG2 is neuron-glial antigen 2. In some aspects of the invention, ONLR-NPCs also express one or more of vimentin and BDNF. Thus, in certain aspects of the invention, the EVs are obtained from ONLR-NPCs that are:

$$nestin ( + ) , SOX 2 ( + ) , GFAP ( + ) , HOPX ( + ) , NG 2 ( - ) , vimentin ( + ) ; or$$

$$nestin ( + ) , SOX 2 ( + ) , GFAP ( + ) , HOPX ( + ) , NG 2 ( - ) , BDNF ( + ) ; or$$

$$nestin ( + ) , SOX 2 ( + ) , GFAP ( + ) , HOPX ( + ) , NG 2 ( - ) , vimentin ( + ) ; or$$

$$nestin ( + ) , SOX 2 ( + ) , GFAP ( + ) , HOPX ( + ) , NG 2 ( - ) , BDNF ( + ) ; or$$

$$nestin ( + ) , SOX 2 ( + ) , GFAP ( + ) , HOPX ( + ) , NG 2 ( - ) , vimentin ( + ) , BDNF ( + ) .$$

Alternatively, or in addition, the ONLR-NPCs can be defined based on expression of one or more of the soluble proteins secreted by the cells and provided in Table 1.

The ONLR-NPCs can also be defined as neural cells that secrete at least 1, 2, 3, 4, 5, 6, 7, 8, 9 or each of the growth factors provided in Table 1. In one aspect, the ONLR-NPCs secrete each of the growth factors provided in Table 1. In one aspect of the invention, the ONLR-NPCs are nestin(+), SOX2(+), GFAP(+), HOPX(+), NG2(−) neural cells that secrete at least 1, 2, 3, 4, 5, 6, 7, 8, 9 or each of the growth factors provided in Table 1.

TABLE 1
Secreted factors produced by ONLR-NPCs

Ciliary Neurotrophic Factor (CNTF)
Fibroblast Growth Factor 1 (FGF-1)
Fibroblast Growth Factor 2 (FGF-2)
Insulin-like Growth Factor 2 (IGF-2)
Midkine (Neurite Growth-promoting Factor 2)
Platelet-derived Growth Factor alpha (PDGFA)
Platelet-derived Growth Factor C (PDGFC)
Transforming Growth Factor-Beta 1 (TGF-β1)
Transforming Growth Factor-Beta 2 (TGF-β2)
Vascular Endothelial Growth Factor A (VEGFA)

The ONLR-NPCs may be further characterized as positionally isolated and residing within the ONLR (see FIGS. 1 and 2), but exhibiting nestin(+)/SOX2(+)/NG2(−) characteristics, unlike the cells in the rest of the eye and optic nerve. ONLR-NPCs of the invention may be additionally characterized as exhibiting an extended ‘starlike’ morphology in living animals.

The ONLR-NPCs may also be characterized based on their ability to express at least 1, 2, 3, 4, 5 or each of the proteins listed in Table 2.

TABLE 2
Additional proteins produced by ONLR-NPCs

Dynein Regulatory Complex Subunit 1 (DRC1)
Dynein Regulatory Complex Subunit 3 (DRC3)
Modulator of VRAC current 1 (MLC1)
Inhibitor of DNA binding 4 (Id4)
Glutathione S-Transferase Alpha 1 (GSTα1)
HOP Homeobox protein (HOPX)

### ONLR-NPC-Secreted Soluble Proteins

The soluble proteins secreted by ONLR-NPCs that may be used in the formulations, compositions and methods of the invention may include 1, 2, 3, 4, 5, 6, 7, 8, 9 or each of the growth factors provided in Table 1. In some instances, the formulations, compositions and methods of the invention include CNTF and FGF2. The soluble proteins may be a collection of one or more soluble proteins isolated from ONLR-NPCs, or a collection of one or more soluble proteins known to be present in ONLR-NPCs but produced by means other than the ONLR-NPCs, and combinations thereof.

### ONLR-NPC EVs

The extracellular vesicles (EVs) of the invention are EVs produced by and/or isolated from ONLR-NPCs, as defined herein. The ONLR-NPC EVs are expected to encompass proteins and nucleic acids. The proteins include one or more growth factors selected from those shown in Table 3. The proteins may also include one or more of the proteins shown in Table 4.

TABLE 3
Factors present in EVs

Alpha-2 macroglobulin
Angiotensinogen, an Angiotensinogen peptide fragment, or Angiotensin
Antithrombin 3-Serpin C1
CD9
CD81
Ceruloplasmin
Insulin-like Growth Factor-1 (IGF-1)
Leucine rich repeat LGI family member 4 (LGI4)
Pigment Epithelium-derived Factor (PEDF)
Osteogenic growth factor 1 (OSTF1)
Thrombospondin-1
Adipocyte Enhancing Binding Protein-1 (AEBP1)

TABLE 4
Additional proteins present in EVs

Cell Growth Regulator with Ring Finger Domain 1
Dynein Regulatory Complex Subunit 3
Growth Arrest and DNA Damage-inducible Protein GADD45 beta
Platelet-derived Growth Factor D/Spinal Cord-Derived Growth Factor B
Transforming Growth Factor beta-1 Protein
Transforming Growth Factor beta-1-induced Transcript 1 Protein
UNC-13 Homolog b

### ONLR-NPC Conditioned Media

The invention is also directed to ONLR-NPC conditioned media containing ONLR-NPC-secreted soluble proteins and/or ONLR-NPC-produced EVs. Such media is cell culture media in which ONLR-NPCs have been grown, such as an in vitro cell culture of ONLR-NPCs, into which soluble proteins and/or EVs have been released. Because ONLR-NPCs secrete soluble proteins and EVs, the media in which the cells are grown becomes conditioned by the presence of these factors.

As used herein, ONLR-NPC conditioned media is any cell culture media in which ONLR-NPC-secreted soluble proteins are present, or ONLR-NPCs EVs are present, or both are present. In some aspects, ONLR-NPC conditioned media is cell culture media in which ONLR-NPCs have been present for at least 12, 24, 36, 48, 60, 72 or 84 hours, or more.

ONLR-NPCs are typically grown in Neurobasal™ (ThermoFisher Scientific) medium supplemented with FGF-2 and -4, and B27 supplementation, and grown on enhanced extracellular matrix (matrigel) at concentrations typically exceeding 10% media (e.g., 10-15%). ONLR-NPCs are typically grown under conditions of enriched CO₂ (3-5%), oxygen concentrations ranging from 5-21%, and 95% relative humidity. In order to induce EV released from the cells, the cells may be cultured under conditions of either ischemic stress (defined as oxygen range from 1-15%, high levels of lactic acid, and glucose levels below 4 millimolar), high levels of sodium bicarbonate (26 mM). However, the skilled artisan will understand that variations in the identity and amounts of the components in such media, and the culture conditions, can be made without altering the health or growth the ONLR-NPCs, or the EVs released by the cells into the media. It should therefore be understood that the identity of the media and conditions in which the ONLR-NPCs are cultured is not critical and that it can vary, but must be one into which soluble proteins and/or EVs are released, if the media is to be an ONLR-NPC conditioned media containing ONLR-NPC-secreted soluble proteins, or ONLR-NPC-produced EVs, or both. In a preferred aspect, the media does not include any animal sourced materials (ASM) and is thus ASM-free.

ONLR-NPC conditioned media may be collected from cultures of ONLR-NPCs through techniques well known in the art, such as simply decanting the media into a separate vessel. ONLR-NPC conditioned media may be stored, such as at 4° C. or frozen, or used immediately. ONLR-NPC conditioned media may be processed to concentrate the media and/or to remove the secreted factors from the media. Suitable processes include concentrating the decanted media, following by dialysis and/or gel filtration to eliminate both uninvolved medium, ions and large molecular weight compounds, such as bovine serum albumen, while retaining EVs.

### ONLR-NPC EV Lysates

The invention is also directed to ONLR-NPC EV lysates. The ONLR-NPC EV lysates of the invention comprise all components from a single ONLR-NPC EV or from a population of ONLR-NPC EVs.

ONLR-NPC EV lysates are produced by rupturing EV membranes and collecting all of the resulting components. ONLR-NPC EV lysates may be prepared by collecting the ONLR-NPCs grown in appropriate medium through centrifugation, washing, and disruption of the cells, followed by isolation of the EVs from the disrupted cells. Other means include isolating culture media from ONLR-NPC cultures, and then separating EVs from the culture media using techniques included centrifugation and washing. The collected EVs are then lysed using means including mechanical, ultrasonic, physical, enzymatic, chemical and osmotic mechanisms. Mechanical mechanisms include repeated freeze/thaw cycles, sonication, pressure, and filtration. Chemical mechanisms include detergents such as Triton X-100. Osmotic mechanism include subject the cells to hypo- or hyper-tonic environments.

### ONLR-NPC EV Extracts

The invention is also directed to ONLR-NPC EV extracts. These EV extracts may be a collection of selected factors isolated from EVs, or a collection of one or more selected factors known to be present in ONLR-NPC EVs but produced by means other than the ONLR-NPCs and combined to form an “artificial” ONLR-NPC extract, and combinations thereof.

ONLR-NPC EV extracts may be produced from ONLR-NPCs by first rupturing cellular membranes, through such means as described herein, and then collecting or isolating selected EV factors, such as one or more growth factors.

ONLR-NPC EV extracts may also be produced by preparing an “artificial” ONLR-NPC EV extract that comprises selected EV factors, such as growth factors (e.g. one or more of the growth factors listed in Tables 1 and 3 (i.e. CNTF, FGF-1, FGF-2, IGF-2, Midkine, PDGFA, PDGFC, TGF-β1, TGF-β2, VEGFA, Alpha-2 macroglobulin, Angiotensinogen, an Angiotensinogen peptide fragment, Angiotensin, Antithrombin 3-Serpin C1, CD9, CD81, Ceruloplasmin, Insulin-like Growth Factor-1 (IGF-1), LGI4, Pigment Epithelium-derived Factor (PEDF), Osteogenic growth factor 1 (OSTF1), Thrombospondin-1, and Adipocyte Enhancing Binding Protein-1 (AEBP1)) or nucleic acids (such as miRNA) produced by means other than the ONLR-NPCs, such as by collecting the selected factors from media of cells engineered to produce the selected factors. The “artificial” ONLR-NPC EV extracts of the invention include (i) those having one or more of the growth factors of Table 3, (ii) those having one or more of the proteins of Table 4, and (iii) those having one or more of the growth factors of Table 3 and those having one or more of the proteins of Table 4. An exemplary artificial ONLR-NPC EV extract is an artificial extract comprising each of Fibroblast Growth Factor 2 (FGF-2); Ciliary Neurotrophic Factor (CNTF); Transforming Growth Factor-Beta 2 (TGF-β2); one or more of Angiotensinogen, an Angiotensinogen peptide fragment, and Angiotensin; Alpha-2 macroglobulin; Thrombospondin-1; Ceruloplasmin; and Pigment Epithelium-derived Factor (PEDF).

Means for producing and isolating the selected factors are well known to the skilled artisan and will vary depending on the identity of the selected factor(s) to be collected or isolated, and the manner in which they are produced. However, suitable means include centrifugation, filters and columns to screen materials based on size, antibody coated chips, plates and columns to isolate peptides/proteins, for example, based on binding affinity.

Additionally, exosomal-identified factors, e.g. the ONLR-NPC EV extracts of the invention, may be encapsulated into liposomes to create an artificial EV-like environment containing the selected ingredients in an artificial EV.

### Methods of Treatment

The invention is also directed to methods of (a) treating or preventing optic nerve diseases, (b) enhancing retinal ganglion cell (RGC) survival, and/or (c) enhancing RGC neurite outgrowth. Because ONLR-NPC EVs are thought to play a role in supporting glial cell growth and development, and by extension, myelination of axons as they emerge from the eye, ONLR-NPC EVs and the factors they contain are likely to have activity in treating or preventing optic nerve hypoplasia, regional axonal dysfunction and hypomyelination. ONLR-NPC EVs and the factors they contain may also enable glial cell replacement and remyelination, enhancing retinal ganglion cell (RGC) survival, and/or (c) enhancing RGC neurite outgrowth.

The methods of (a) treating or preventing optic nerve diseases, (b) enhancing retinal ganglion cell (RGC) survival, and/or (c) enhancing RGC neurite outgrowth of the invention comprise administering to a subject in need thereof a therapeutically effective amount of a composition comprising one or more of.
(i) ONLR-NPC-secreted soluble proteins,
(ii) a population of ONLR-NPC-produced EVs,
(iii) ONLR-NPC conditioned media containing ONLR-NPC-secreted soluble proteins and/or ONLR-NPC-produced EVs,
(iv) ONLR-NPC EV lysates, and
(v) ONLR-NPC EV extracts,
   thereby (a) treating or preventing optic nerve diseases, (b) enhancing retinal ganglion cell (RGC) survival, and/or (c) enhancing RGC neurite outgrowth. Together, the compositions of (i)-(v) are termed “ONLR-NPC-based compositions” herein. The ONLR-NPC EV extract of (v) may in the form of an artificial EV. An exemplary artificial ONLR-NPC EV extract is an artificial extract comprising each of Fibroblast Growth Factor 2 (FGF-2); Ciliary Neurotrophic Factor (CNTF); Transforming Growth Factor-Beta 2 (TGF-β2); one or more of Angiotensinogen, an Angiotensinogen peptide fragment, and Angiotensin; Alpha-2 macroglobulin; Thrombospondin-1; Ceruloplasmin, and Pigment Epithelium-derived Factor (PEDF).

Optic nerve diseases that may be treated using the methods of the present invention include, but are not limited to, open-angle glaucoma, such as primary open-angle glaucoma (POAG), angle-closure glaucoma, optic nerve hypoplasia, optic nerve hypomyelination, regional axonal dysfunction, nonarteritic anterior ischemic optic neuropathy (NAION), and optic neuritis.

In one aspect, the composition administered to the subject is a pharmaceutical formulation comprising a pharmaceutically acceptable carrier and one or more of the ONLR-NPC-based compositions of the invention.

In one aspect, the ONLR-NPCs from which the soluble proteins and EVs are obtained are characterized based on their ability to secrete one or more factors selected from the group consisting of Nerve Growth Factor (NGF), Latent Transforming Growth Factor-Beta 1 (TGF-01), Fibroblast Growth Factor 1 (FGF1), Fibroblast Growth Factor 2 (FGF2), Vascular Endothelial Growth Factor (VEGF), Mesenchymal Astrocyte Neurotrophic Factor (MANF), Connective Tissue Growth Factor (CTGF), Insulin-like Growth Factors-1 and -2 (IGF-1 and IGF-2), and Ciliary Neurotrophic Factor (CNTF). In another aspect, the ONLR-NPCs are characterized based on their ability to produce one or more of the proteins listed in Table 2. In further aspect, the cells secrete one or more of the factors of Table 1 and produce one or more of the proteins of Table 2.

In each of the methods of the present invention, a “therapeutically effective amount” of an ONLR-NPC-based composition is administered to a subject. The effective amount will vary between subjects and the goal of the method, e.g. the identity of the optic nerve disease being treated. However, the effective amount is one that is sufficient to achieve the aim or goal of the method.

In the methods of the invention, the ONLR-NPC-based composition may be administered to the eye of the subject, such as via topical application to the surface of the eye (e.g. eye drops), via subconjunctival injection, via intravitreal injection into the interior of the eye, or via retrobulbar injection into the space behind the globe of the eye. Such injections include depot injection through the conjunctiva or depot injection behind the globe of the eye. There may also be applications using nanoparticles that, when injected intravenously, are directly taken up by the cells of the optic nerve head, or by administration of the genes or messenger RNAs of the individual factors that have been placed in a vector for direct administration by intravitreal administration.

Administration of the ONLR-NPC-based composition may be via any of the means commonly known in the art. When administered to the eye, such routes include topical and intraocular, subconjunctival and retrobulbar injections. When administered to sites other than the eye, such routes include intravenous, intraperitoneal, intramuscular, subcutaneous and intradermal routes of administration, as well as topical, nasal application, by inhalation, orally, rectally, vaginally, or by any other suitable mode.

The pharmaceutical compositions of the present invention may be formulated, for example, for topical, intraocular, oral, sublingual, intranasal, rectal, transdermal, mucosal, pulmonary, or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of an ONLR-NPC-based composition can be used to effect such administration. In preferred aspects on the invention, the ONLR-NPC-based composition is administered to the subject as a topical drop for ophthalmic administration.

Appropriate doses and dosing schedules can readily be determined by an attending physician without undue experimentation depending on the characteristics of the subject to be treated and the identity of the optic nerve disease. Administration frequencies for the compositions of the present invention include 4, 3, 2 or once daily, every other day, every third day, every fourth day, every fifth day, every sixth day, once weekly, every eight days, every nine days, every ten days, bi-weekly, monthly and bi-monthly. The duration of treatment will be based on the optic nerve disease being treated and will be best determined by the attending physician.

In the methods of the invention, the ONLR-NPC-based composition administered to the subject may be in the form of a pharmaceutical formulation comprising (a) one or more of the ONLR-NPC-based compositions and (b) a pharmaceutically acceptable carrier.

Suitable examples of carriers are well known to those skilled in the art and include water, water-for-injection, saline, buffered saline, dextrose, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, nano-particles, and liposomes. The terms specifically exclude cell culture medium. The formulations may further comprise stabilizing agents, buffers, antioxidants and preservatives, tonicity agents, bulking agents, emulsifiers, suspending or viscosity agents, inert diluents, fillers, and combinations thereof.

The subject may be a human, a non-human primate, bird, horse, cow, goat, sheep, a companion animal, such as a dog, cat or rodent, or other mammal.

### III. Examples

### Protocol 1. Confirming Presence of NPCs in ONLR

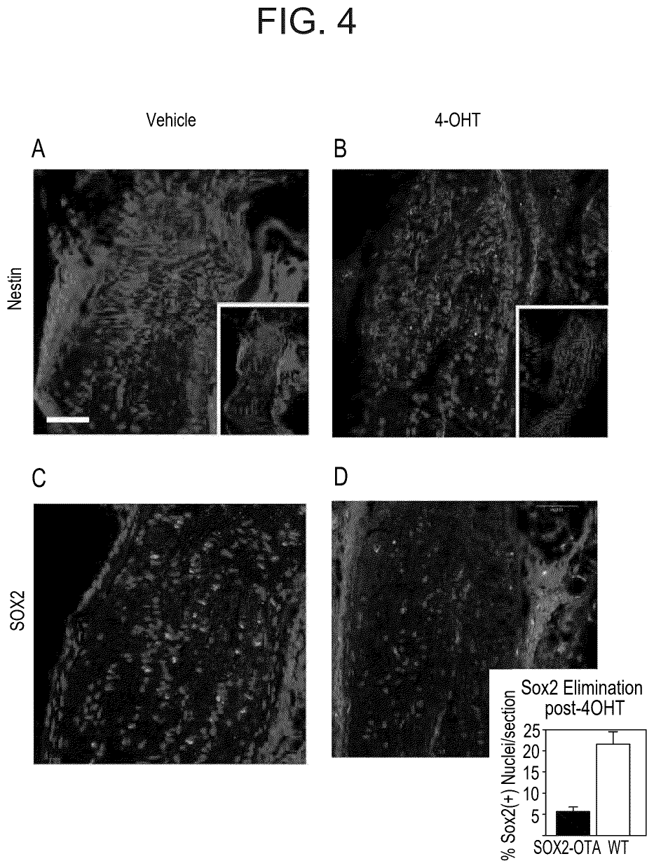

ONLR-NPCs strongly express Lhx2, HOPX and nestin, while oligodendrocyte progenitors (OPCs) and oligodendrocytes in the ON do not [43]. ONLR-NPCs also strongly express SOX2 at higher levels than do OPCs or oligodendrocytes. Utilizing a mouse transgenic approach, the SOX2-ER2-Cre transgene enables SOX2-directed expression of the Cre recombinase only following permissive binding of the modified estrogenic compound 4-hydroxytamoxifen (4OHT), tamoxifen's active metabolite. Cross breeding mice with the SOX2-ER2-Cre transgene with animals possessing the mutant gene construct ROSA 26-(LoxP)-neo-Stop-(LoxP)-(Diphtheria toxin antigen A (DTA)) generates a double mutant animal (SOX2-Cre×ROSA-DTA). This strain will only express DTA after tamoxifen or 4OHT administration, allowing for normal growth and development. Following tamoxifen or 4OHT administration, these double mutants will express diphtheria toxin A (DTA) in all cells currently expressing the SOX2 gene, killing them and resulting in selective ablation of SOX2-expressing cells (FIG. 4). Systemic tamoxifen administration is lethal to the animal within one week. Control (WT) mice reveal that the doses used for selective DTA expression in double mutant animals do not cause ON damage/myelin loss after treatment with RB 4OHT, compared with vehicle. This preliminary data reveals that retrobulbar injection of 4OHT in SOX2-Cre×ROSA-DTA animals results in selective loss of SOX2/nestin (+) ONLR-NPCs (FIGS. 4B and D), while there is no loss on the contralateral (vehicle treated) side (FIGS. 4A and C).

Previous studies failed to identify any stem cell component in the ONL-retina/ON junction. Typically, neural stem cells express the intermediate filament protein nestin [65], although other cell types can also express this protein [62]. The high concentration of connective tissue in the ONL typically necessitates tissue-paraffin embedding, which eliminates nestin immunoreactivity. Using frozen section tissue, it was determined that the ONL has a nestin expression gradient in adult mouse eyes (FIG. 4A). Nestin expression is strongest in the region immediately behind the retina (FIG. 4A and in humans, FIG. 3F), and decreases over the anterior ON, reaching a minimal level of expression in the myelinated ON (FIG. 4C; red is nestin, green nuclei are SOX2). This suggested that the ONL-nestin (+) component was in some way related to myelination.

### Protocol 2. Identification ONLR-NPC Secreted, Exosome-Related ON Neuroprotective Elements

Initial experiments have been conducted and further experiments will be conducted to identify the ONLR-NPC secreted, exosome-related ON neuroprotective elements. Use has been and will be made of human donor cell cultures, gene sequencing, and tandem Liquid Chromatography-mass spectroscopy based protein analysis (LC-MS/MS), coupled with isolated rodent RGC cultures, to determine:
a. miRNAs and growth factors associated with ONLR-NPC-secreted exosomes, and
b. specific miRNAs and proteins responsible for RGC-neuroprotective effects.

### NPC Analysis

Initial experiments included the following. Deep sequencing was performed on mRNA from isolated macular retina, ONLR, and distal ON from three young (9 y/o) male and female old world rhesus macaque primates (NHPs) euthanized as part of nonvisual experiments, without ocular conditions and whose ONLR-NPCs would reasonably be expected to be unaffected (no chemotherapy or cerebral spinal radiation). ONLR tissue was also isolated from six outbred Sprague-Dawley rats and deep sequencing was performed for expression comparison with the results seen with primates. Post-sequencing analysis identified >27,800 sequences expressed by the primate ONLR and retina, and >22,200 for rat ONLR. Raw counts were generated by htseq-count. Individual gene sequences were corrected for gene length bias by transcripts per kilobase million (TPM). The maximum signals were ˜18 (opsin in retina, MBP in ON). A minimum cutoff for significance was set at 2.0. In the monkey tissue, 627 miRNAs were identified, with signal >2.0. ONLR was evaluated for differential gene expression (DEG) by the formula: cutoffs=false discovery rate (FDR)<0.05, with DEGs=genes with log fold change (LFC) >±1.0 with mean ONLR counts/(mean retina+mean ON counts). Table 5 summarizes results.

TABLE 5
Differentially expressed genes summary (DEG): The numbers of
genes differentially expressed for each comparison based on
filter cutoffs of FDR and log fold change (LFC) >= +/−1

| | | LFC_{—} | LFC_{—} | Read | | | Total |
|---|---|---|---|---|---|---|---|
| Comparison | FDR | up | down | count % | Up | Down | DEG |
| Lam/Ret + | 0.05 | 1 | −1 | 0.01 | 163 | 140 | 303 |
| ON | | | | | | | |

303 differentially expressed genes were identified in the analysis, with 163 genes increased in the ONLR and 140 genes decreased (Table 5). Increased DEGs included a number of growth factors and related proteins (GDF5, FGF3, IGFBP4, NENF). A significant number of other DEGs were associated with exosomal function (Table 6).

TABLE 6
Selected ONLR differentially expressed genes. The LFC-log fold ratio
is the ONLR expression divided by both ON and retina expression.
No differential expression = 0.5. Only those genes that have
an FDR <0.5 are listed. Known gene functions are indicated.

| | ONLR | Log2 fold | FDR | | |
|---|---|---|---|---|---|
| | Expression | change ONLR/ | Significant | | |
| Gene | (TPM) | Retina + ON | (ONLR) | Function | Notes |
| Unc13c | 15.159 | 3.5 | P < 0.05 | Exosome | Considerably elevated |
| | | | | protein | only in ONLR |
| CD81 | 15.255 | 1.29 | P < 0.05 | Exosome | High in ONLR and ON |
| | | | | protein | |
| CD63 | 14.493 | 1.31 | P < 0.05 | Exosome | High in ONLR and ON |
| | | | | protein | |
| Anxa1 | 11.120 | 2.28 | P < 0.05 | Exosome | High in ONLR and ON |
| | | | | protein | |
| Mir675 | 7.870 | 3.32 | P < 0.05 | MicroRNA | Neuroprotective |
| Mir195 | 4.157 | 4.1 | P < 0.05 | MicroRNA | Neuroprotective |
| Mir143 | 6.400 | 3.38 | P < 0.05 | MicroRNA | Increase reduces |
| | | | | | neuroprotection |

A number of microRNAs (Mir's) associated with neuroprotection following CNS trauma or ischemia were also identified in the exosome analysis of rat ONLR-NPCs (Table 7). These perform different functions such as neuroprotection by increasing brain derived neurotrophic factor (BDNF) (rat microRNA (rno-) MiR 30a-5p, Mir 10b-5p), neuroprotection in combination with other MiRs (Mir 92a-3p), regulating TGFβ forms (MiR 143-3p), as well as increasing neurite/axonal regrowth (MiR Let 7f-5p, Let-7a-5p, MiR 7a-5p), suppressing inflammation (MiR 125-1-3p, MiR let-7c-5p, Mir 146a-5p, Mir 21-5p, Mir 216a-5p), and enhancing blood brain barrier stability (Mir 224-5p, Mir 320-3p, Mir 27a-3p). These are shown in Table 7. Reference numbers are shown in the right most column (ref).

TABLE 7

| MiRNA species | | | |
|---|---|---|---|
| found in ONLR- | Mean | | |
| NPC exosomes | expression | std dev | Mir-TARbase and Targetscan |
| rno-let-7f-5p | 78184.39 | 17640.17 | Neurite regenerative (8528) Anti- |
| | | | inflammatory/anti-apoptotic (7865) |
| rno-let-7c-5p | 49812.88 | 10476.18 | Anti-inflammatory (7429) |
| rno-let-7a-5p | 37470.29 | 6439.81 | Neurite regenerative (8528) |
| rno-miR-92a-3p | 1906.17 | 358.80 | RGC Neuroprotective in combination (7868) |
| rno-miR-146a-5p | 28608.40 | 2684.59 | Anti-inflammatory (7429) |
| rno-miR-21-5p | 28444.57 | 5381.57 | Anti-inflammatory (7429) |
| rno-miR-101a-3p | 2386.13 | 184.07 | Anti-apoptotic; Targets JNK |
| rno-miR-125b-1- | 1426.89 | 136.63 | Anti-inflammatory by suppressing STAT3 |
| 3p | | | |
| rno-let-7c-5p | 49812.89 | 10476.19 | Anti-inflammatory (7429) |
| rno-miR-10b-5p | 988.41 | 185.56 | upregulates BDNF (Muller cells) |
| rno-miR-30a-5p | 19964.03 | 4014.91 | upregulates BDNF (Muller cells) |
| rno-miR-143-3p | 16372.52 | 1249.97 | Regulates TGFb (7426) |
| rno-miR-224-5p | 664.45 | 256.32 | preserves BBB integrity by decreasing BBB |
| | | | permeability (7564) |
| rno-miR-320-3p | 3093.00 | 907.31 | Preserves BBB integrity as 320a (7564) |

Mir 143 and Mir 320a-3p, these are found in the ONLR-NPC exosome cultures and know to be elevated in monkey ONHr by deep sequencing.

These results support the hypothesis that ONLR-NPCs play a key role in RGC (i.e. CNS) axonal neuroprotection and resistance to ischemic or other stress, and that this effect could be at least partly communicated via ONLR-NPC secreted exosomes.

### Protocol 3. Neuroprotective Effects of ONLR-NPC-Secreted Exosomes and Components in In Vivo Model of ON Ischemia

Experiments have been conducted to determine whether ONLR-NPC-secreted exosomes and their components are directly neuroprotective in an in vivo model of ON ischemia.

Exosomes from Human ONLR-NPCs Directly Neuroprotect RGCs and Enhance Neurite Growth

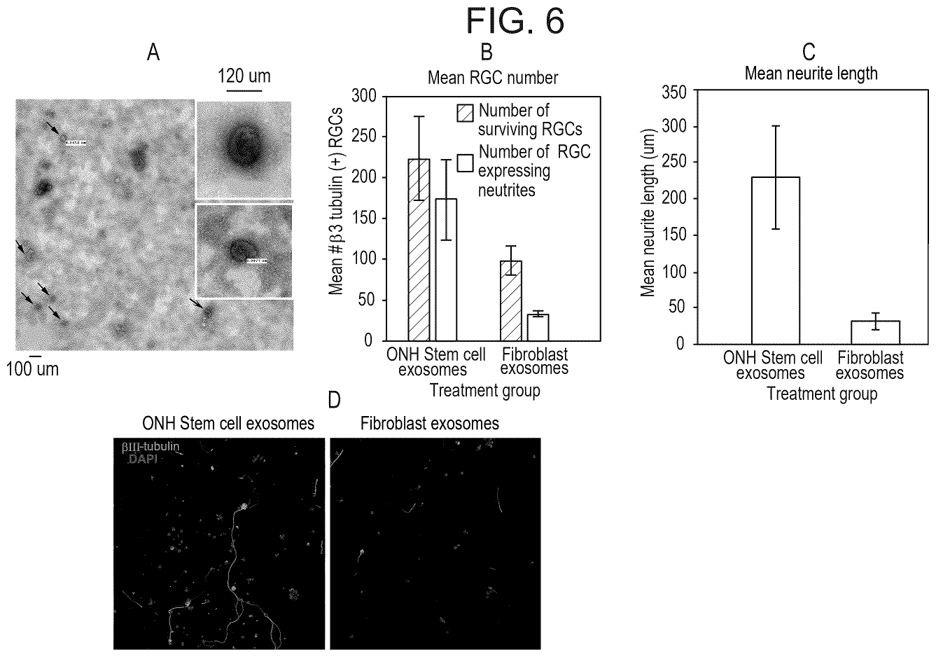

Many factors have been identified as RGC-neuroprotective, but few enhance both RGC survival and improve axonal regeneration. It is hypothesized that ONLR-NPC secrete exosomes that might provide both neuroprotection during stress and enable RGC-axonal recovery after stress. Human donor ONLR-NPC cultures were generated under an UMB-IRB exemption, through an agreement with the Living Legacy Foundation (LLF). This has enabled living ONLR tissue to be obtained within 1½ hours of aortic cross clamping, from donors ranging from 12-50 years of age. ONLR-NPCs have been consistently cultured from donors <41 years of age (total number of donors to date=8; Total number of donors younger than 41 y/o=5), consistent with an earlier report by the inventors [8] that human ONLR-aNPCs decline significantly after age 40. Human ONLR-aNPCs express nestin and SOX2, and form neurospheres in low adhesion medium. Conditioned medium from human ONLR-aNPCs (3rd-5th subcultures; 50-60% confluent) were evaluated both for exosome secretion and function. Exosomes were extracted from this medium using the high speed centrifugation procedure [44](TEM analysis: FIG. 6A). Exosomes were analyzed from human ONLR-NPC conditioned medium for their ability to enhance RGC survival and neurite outgrowth in isolated rat RGC cultures and functional assays were compared against exosomes secreted by fibroblasts (FIG. 6B-D).

Widely used models of rodent and primate NAION (rNAION and pNAION) were generated [45,46] and these models were utilize to assay neuro-protective agents and regenerative strategies [47-50]. These models have allowed: 1) identification and characterization of ONLR-NPCs from humans and rodents; 2) discovery that the human ONLR differentially expresses exosome proteins, compared with other ocular regions; 3) identification of likely exosome-associated candidates for neuroprotection and regeneration; 4) demonstration that ONLR-NPCs express exosomes that both enhance RGC survival and neurite extension (the combination of these two factors is very unusual); 5) development of appropriate tools to identify the components of human ONLR-NPC exosomes and to determine whether human ONLR-NPC-derived exosomes and their components can enhance RGC survival and regeneration after optic nerve ischemia.

### Protocol 4. Neuroprotective Effects of ONLR-NPC-Secreted Exosomes in Glaucoma Model.

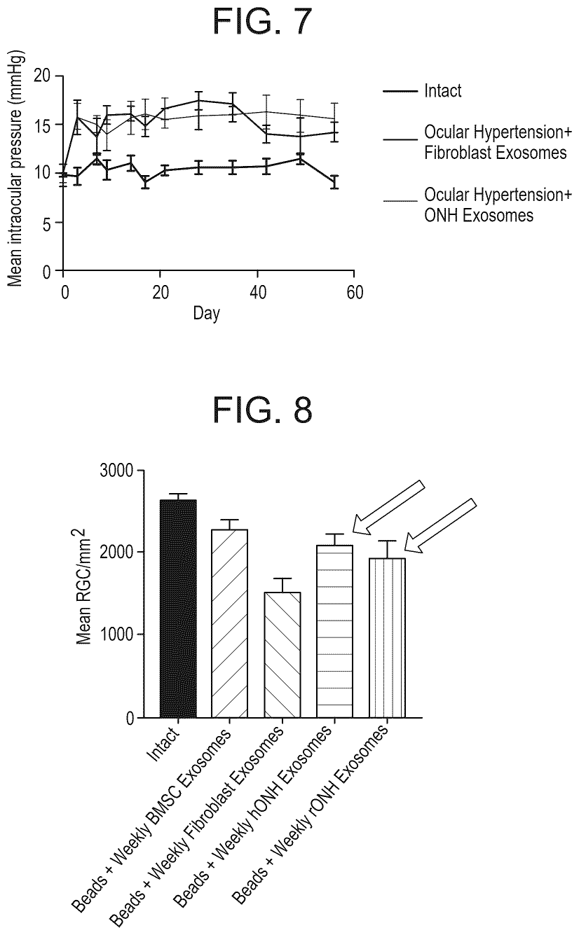

Human ONLR-NPC secreted exosomes have already been shown to be effective in a rat model of glaucoma (FIGS. 7 and 8). In this model, sterile 6 μm and 10 um polystyrene microspheres were aseptically injected into the anterior chamber of a rat eye to fill the anterior chamber and block normal aqueous humor outflow, resulting in an increase in intraocular pressure (FIG. 7, red and green lines; compare with control/no microspheres, blue line). Following glaucoma induction, FIG. 8 shows results of quantified RGC survival when the eyes of the animal were intravitreally injected either with exosomes purified from bone marrow stem cells (BMSCs), exosomes purified from fibroblasts, exosomes purified from human ONLR-NPCs (hONH), or exosomes purified from rat ONLR-NPCs (rONH). There is a strong increase in RGC survival in animals treated with either human or rat ONLR-NPC exosome preparations.

### Protocol 5. Single Cell Sequencing

Optic nerve heads (1 mm length measured from the eye to the end of the stump) from 12-20 day old rat pups were isolated and pooled in ice-cold hibernate buffer A (Thermo-Fisher). Tissue was triturated using a sharp scalpel blade. The macerated tissue was then digested using a Miltenyi single cell isolation kit with papain, and the tissue bits further dissociated using trituration through a 500 micron size siliconized pipette end. Following dissociation, the tissue was filtered through a 70 micron mesh filter and then purified cells were isolated through a discontinuous idodixanol (optiprep) gradient. Pelleted cells were then counted and complementary DNA (cDNA) from individual cell mRNAs were generated and barcoded using the 10× genomics platform. Barcoded cDNAs were then analyzed using the core sequencing facility at Johns Hopkins University.

### Protocol 6. Retinal Ganglion Cell Assay for EV Activity

Extracellular vesicles were isolated from conditioned culture medium obtained from optic nerve laminar neural progenitor cells (ONLR-NPCs), and from rat primary fibroblasts. The cell cultures were generated from rat optic nerve heads (1 mm each-NPCs) and rat skin (fibroblasts), using standard cell isolation techniques (Miltenyi papain cell dissociation kit). Cell cultures were grown to 65-75% confluency and then serum-free medium (see below) was used to feed the cultures and was collected and pooled for each cell type.

10 ml conditioned media was collected from each culture: (i) rat (ONLR) NPCs grown in serum free NT2 media and (ii) rat skin fibroblasts grown in DMEM F12 supplemented with 10% exosome depleted FBS (INV-A272080) and antimycotic-antibiotic solution. Collected media was spun down at 3000×g for 10 min to eliminate cell debris and larger particles. Conditioned cell growth media was concentrated by using 10,000 MW spin column (SIG-UFC 801024) at 4° C. The solution was then sterilized by running it through a 0.22 m filters (Millex GV SLGV004SL). Collected concentrated and sterilized media was subjected to Exosome isolation by using ExoQuick-TC Ultra EV isolation kit EQULTRA-20TC-1 (System Biosciences) following manufacturer's instructions. The culture medium was collected from the first three subcultures only and stored at −80° C. until EV isolation. Final product protein concentration was measured and the precipitated EV pellets are resuspended in phosphate buffered saline (PBS, pH 7.4) to approximately 2 mg/ml, aliquoted and frozen at −80° C. until use.

Retinal tissue was isolated from 21 postnatal rats, after removing the lens and sclera. The tissue was minced, and then enzymatically digested to generate free cells, using papain and DNAse 1, and incubating for 90 minutes. After the 90 minute incubation the tissue was spun down at 300 g for 5 minutes, and then the cell pellet was resuspended in a solution of 1.35 ml Earles balance salt solution (EBSS), along with 150 microliters of reconstituted albumin ovomucoid inhibitor and 751 of DNase I. The cell suspension was gently layered onto the top of a falcon tube containing 2.5mls of albumin ovomucoid inhibitor to form a discontinuous density gradient, and centrifuged at 70 g force for 6 minutes. All the supernatant was removed, and the pellet was resuspended in 1 ml of neurobasal-A. The cells were counted using a haemocytometer. Additional Neurobasal-A was added to the cell suspension to generate a volume which yielded 125,000 dissociated retinal cells per 300 μl.

The cells were layered into an 8-well chamber slide (125,000 dissociated retinal cells per well) previously prepared for retinal culture by sequentially coating the wells with 100 micrograms/ml poly-D-lysine/60 minutes, washing 2× with phosphate buffered saline (PBS) and then coating the wells with laminin (20 micrograms/ml/30 minutes). Laminin was removed and the retinal cells were added to each well. Sufficient EV from either ONLR-NPCs or primary fibroblasts were then added to each well to generate an EV concentration of 2× that of the original culture medium. Following distribution of the cellular preparation in the wells and EV supplementation, cultures were incubated at 37° C. for three days at 21% O₂ and 5% CO₂. The assay was performed in triplicate (3 wells/condition).

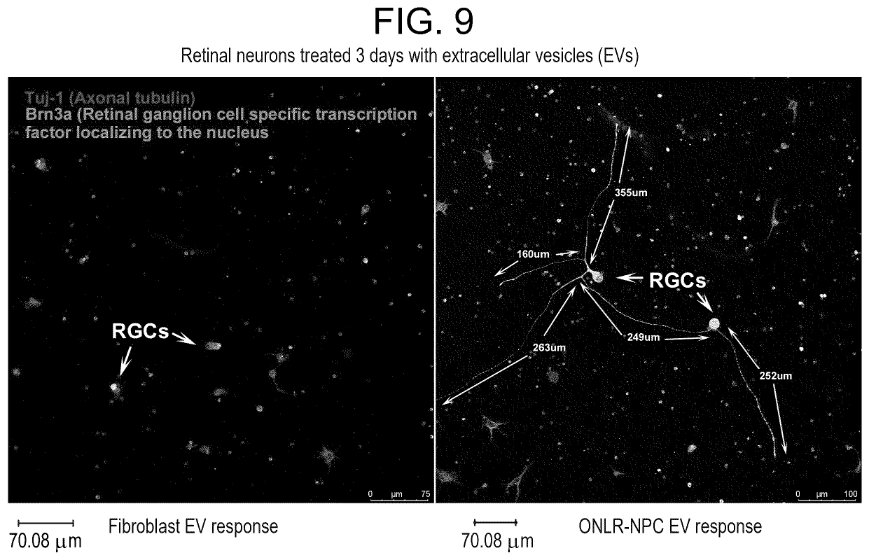

After 3 days, the medium was removed, cells were fixed with 4% paraformaldehyde in PBS (PFA-PBS), and immunocytochemically stained using antibodies to either Brn3a (1:500) or Tuj1 (1:500) (FIG. 9). Retinal ganglion cells (RGCs), whose axons typically comprise the optic nerve, were counted as Brn3a(+) nuclei, and RGC-associated axons are quantified by length (in microns), and number of neurites associated with RGCs.

The results shown in FIG. 9 reveal >2:1 preservation of neuron (RGC) cells and a 360-fold increase in the total length of neurites produced using NPC-derived EVs, compared with fibroblast EVs.

### Protocol 7. Confirmatory Experiments

As discussed below, the RGC-neuroprotective components of ONLR-NPCs were identified utilizing a combinatorial approach of analysis of the ONLR-NPC, exosome/extracellular vesicle, combined with single cell sequencing of the ONLR, and further identification of the ONLR-NPC secretome (all secreted growth factors).

### Rat ONH Single Cell Preparation for Gene Expression

12 ONHs (optic nerve heads; 1 mm long) were obtained from animals of both sexes of a 20 d old litter of SD-rats. Tissue was pooled in ice cold hibernate A (ThermoFisher) and triturated first using an Ophthalmic 75 degree angle microblade, digested by incubating sequentially with papain, followed by papain+DNAse 1 (Miltenyi neural cell dissociation system) as recommended by the manufacturer. The tissue was further triturated by passage through a siliconized polished 500 um bore Pasteur pipette, then filtered through a 70 um mesh screen to obtain single cells. Cells were then re-purified using a discontinuous nonionic Iodixanol density gradient (Optiprep: Millipore Sigma-Aldrich). Following washing and repelleting, cells were refiltered through a 40 um pipette filter, repelleted and then counted using trypan blue. Approximately 20,000 cells were isolated and used in a 10× Genomics Chromium microfluidics platform with a Chromium Next Gem automated single cell 3′ library and gel bead kit V3.1. Following bar coding and cDNA synthesis, cDNA was amplified for 5 cycles. Following clean-up, cDNA was sent for sequencing using the Illumina sequence platform available at Johns Hopkins University. Cell sequence data was then analyzed for cell-specific markers and suboptimal cell data was removed from the final distribution.

### Rat ONLR-NPC-Cultured Medium Preparation

ONLR-NPC mixed cultures were generated from 16-20 isolated PN 20 d rat optic nerve heads (1 mm long). The dissected ONHs contained the site where the ON enters the sclera of the eye. Tissue was pooled in ice cold hibernate A (ThermoFisher) and triturated first using an Ophthalmic 75 degree angle microblade, then digested by incubating sequentially with papain, followed by papain+DNAse 1 (Miltenyi neural cell dissociation system) as recommended by the manufacturer. The tissue was further triturated by passage through a siliconized polished 500 um bore Pasteur pipette, then filtered through a 70 um mesh screen to obtain single cells. Cells were cultured on 12% Matrigel using coated surface plastic well plates and medium consisting of equivalent amounts of F12 and DMEM, supplemented with 5% bovine serum albumen, B-27 and glutamax, along with FGF2 and Insulin. Cells were incubated in 21% O₂ and supplemented with 5% CO₂ at 37° C. Cells were grown to 75% confluency and then split for reculture. Conditioned medium for exosome isolation was collected every three days when the cells were at 65-75% confluency, and pooled and stored at −80° C. until use. For extracellular vesicle (EV) analysis and functional assays, only conditioned medium from the first three subcultures were collected. A maximum of 60 ml conditioned medium was collected for each preparation.

Extracellular Vesicle (EV) Isolation from Cultured Medium for Cell and In Vivo Experiments.

Control EVs were generated from pooled conditioned medium isolated from human and rat ONH cultures, and primary rat fibroblast cultures from the same animals grown for collection in fetal bovine serum (FBS)-free medium, since FBS can also contribute protective EVs. Pooled conditioned medium consisted of medium collected from the first three passages of each cell line

EVs were isolated and concentrated using Exoquick TC (System Biosciences, Palo Alto CA). Purified extracellular vesicles were stored in D-PBS in 100 ul aliquots at −80° C. until use. EVs were used at a ratio of 2:1 original culture medium for cell culture experiments, and 2 ul undiluted aliquots for intravitreal injection and administered to ex vivo dissociated primary retinal cell cultures generated from adult rat retinae (D₀). Long-term storage of EVs resulted in loss of potency; thus, EVs were stored for no more than 3 months prior to use.

EV Isolation from Cultured Medium for Protein and RNA Analyses
EVs were physically characterized as to size via two-color fluorescent nanoparticle tracking analysis (fNTA) using zetaview Quatt (particle metrix). EVs were reacted prior to analysis with plasma membrane deep red dye (CMDR) and fluorescent labeled anti-CD63 antibody.

### EV Proteomic Analysis

Conditioned medium was collected from early (1-3) passage cultures generated from rat ONH. Mean EV sizes for the three different preps with scatter ranged from 157-201 nm, while labeled EV sizes ranged from 161.6 nm/134.4 nm (prep 3) to 177 nm (CMDR)/213 nm (CD63) (prep 1). Proteins from concentrated EVs were identified by the standard method of liquid chromatography-tandem mass spectroscopy/mass spectroscopy (LC-MS/MS) for individual peptides, and concentrations for each culture prep gave a mean relative protein expression. 257 proteins were identified based on unique peptide identification, and in all three preparations. Typical EV-associated proteins were detected at high concentrations, including LGALS3 bp (11/258) and major vault protein (MVP) (35/258). Exosome-associated proteins included CD9 (198/258) and CD81 (125/258). CD63 was expressed at lower levels but detectable by capillary electrophoresis (CE). Expression of CD9- and -81 via CE was also confirmed.

Within the group of EV-associated proteins, multiple peptides with neuroprotective properties were identified. These included Gelsolin, Osteopontin, Colony stimulating factor-1 (CSF1), ceruloplasmin, angiotensin, Thrombospondin, PEDF/Serpin F1, Glutathione S-transferase P and IGF1. However, a number of factors associated with neurodegeneration were also identified, including Periostin, SerpinA3, and Somatomedin B (Vitronectin). Since ONH cell cultures include multiple non-NPC cell types (microglia, vascular components, OPCs, oligodendrocyte) as well as NPCs, it is likely that many of these proteins derive from non-NPC exosomes. Additionally the Matrigel used to establish and maintain these cultures may contribute contaminants, since cultivation of ONLR-NPCs require high matrigel concentrations (10-15%).

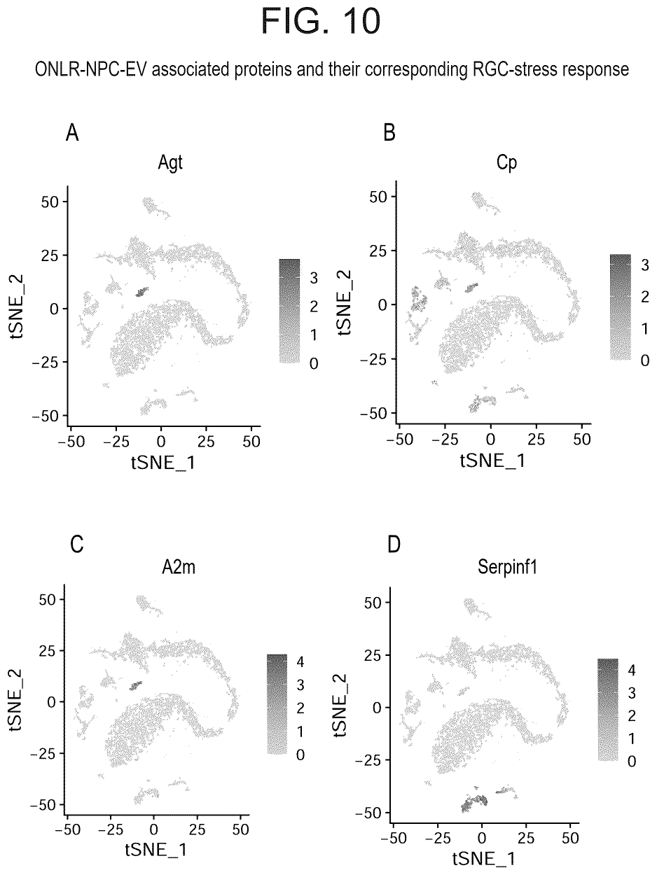

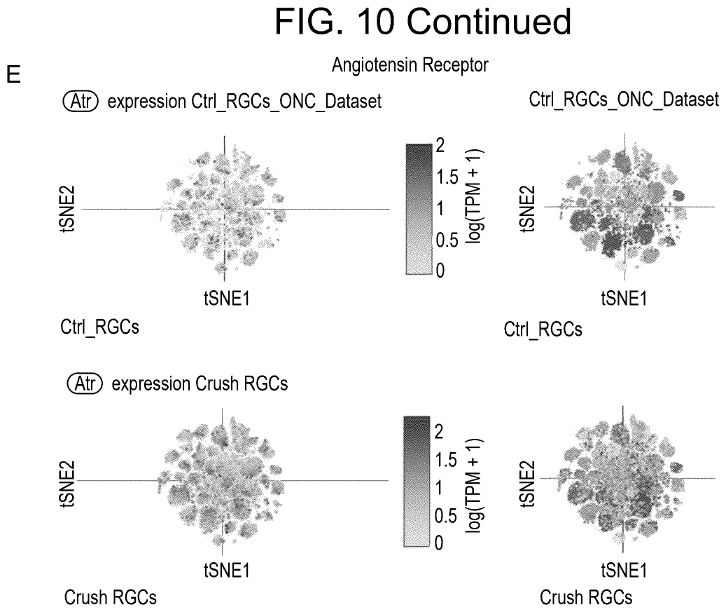

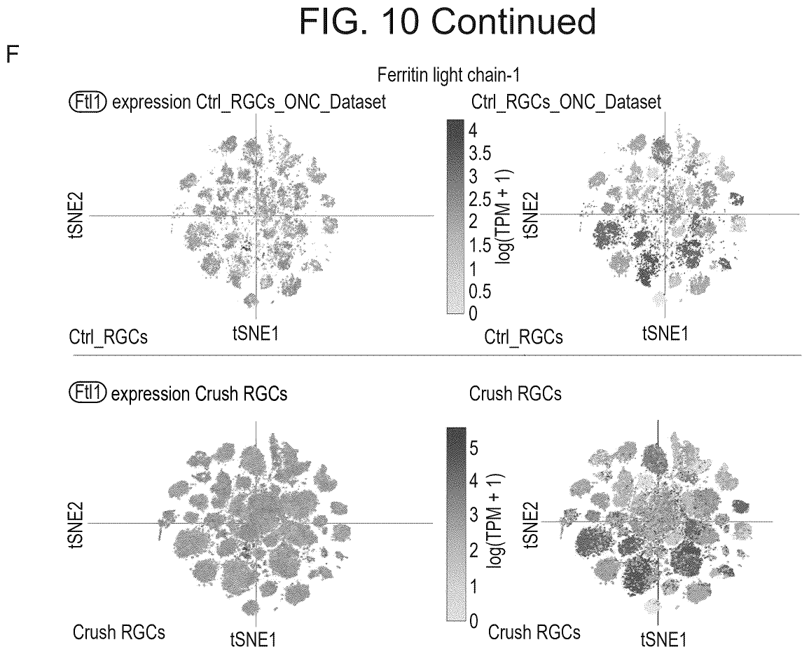

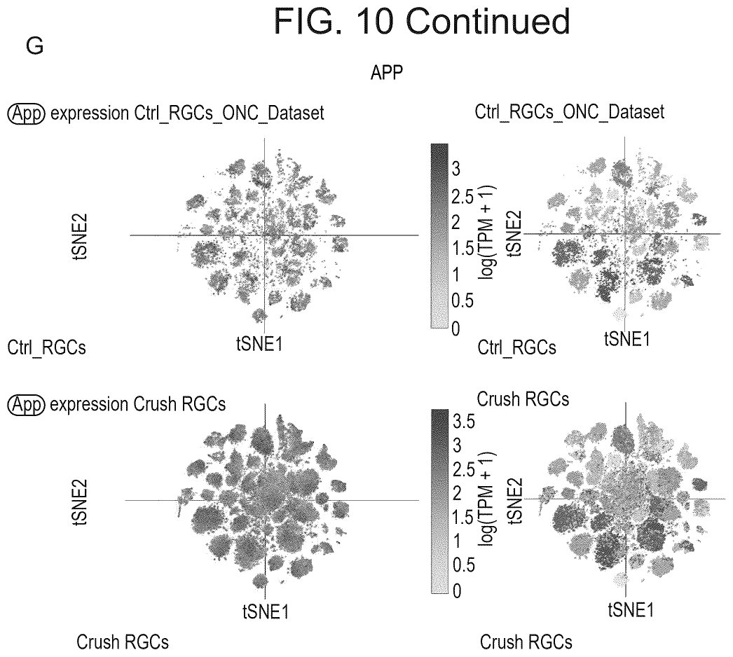

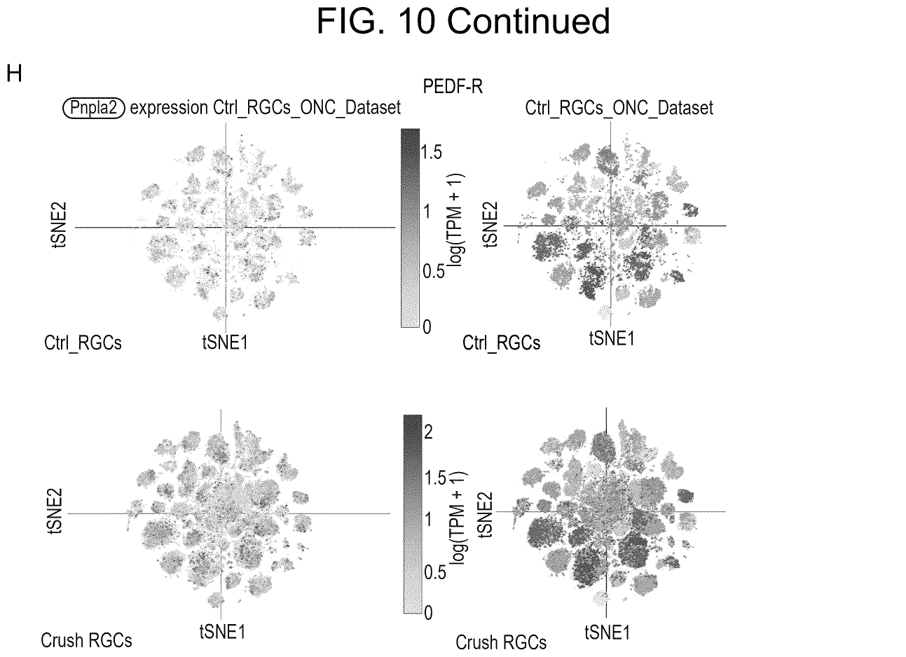

To identify ONLR-NPC-EV specific protein contributions, EV-proteome expression was cross-correlated with ONH sc-seq data. ONLR-NPC EVs exclusively contributed A2M, Cp, and angiotensin, while strongly secreting PEDF/Serpinf1, TF, OSTF1, and Gelsolin, along with a number of other cell types. A similar in silico analysis was then performed using the RGC stress response database, evaluating whether post-stress RGCs increase their intracellular requirement for secreted proteins without known receptors. These results are shown in FIG. 10.

### Single Cell Sequencing Defines the ONLR-NPC Geneset in ONH

ONH sc-seq generated 14 cell clusters via the 10× genomics loupe browser. The data was re-analyzed using SEURAT, which yielded a more developmental-associated orientation with 17 clusters. Elimination of poor-quality cells enabled resolution into 10 distinct cell species in the tissue. The distal portion of the ONH is continuous with the remaining myelinated optic nerve. It therefore contains oligodendrocyte progenitor cells (OPCs), differentiating and mature oligodendrocytes. These cell types express distinct patterns of expression that easily enable identification within the ONH population and show the developmental pathway for these associated cells. NPCs are a class of astrocytes, and thus express high levels of GFAP in the NPC group of cells (not shown) and the homeodomain protein Lhx2 (not shown) and HOPX protein (not shown). ONLR-NPCs are thus a distinct cell group easily distinguished in both analyses. This provided the basis for discerning which cells expressed specific proteins found in EVs, as well as determining ONLR-NPC-expressed directly secreted growth factors that are potentially relevant to RGC stress and survival.

### ONLR-NPCs Support RGC Function by NPC-Specific EV Secretion

NSC/NPC EVs can enhance and support neuronal function. The ability of ONH-EVs from rat ONH and fibroblast cultures to support RGC survival and neurite extension was evaluated. Untreated primary cultures yielded ˜95±5 (sem) Brn3a(+) RGC nuclei per well at 3 d post-plating. EVs derived from fibroblasts yielded similar results (97±8 (sem) RGCs/well).

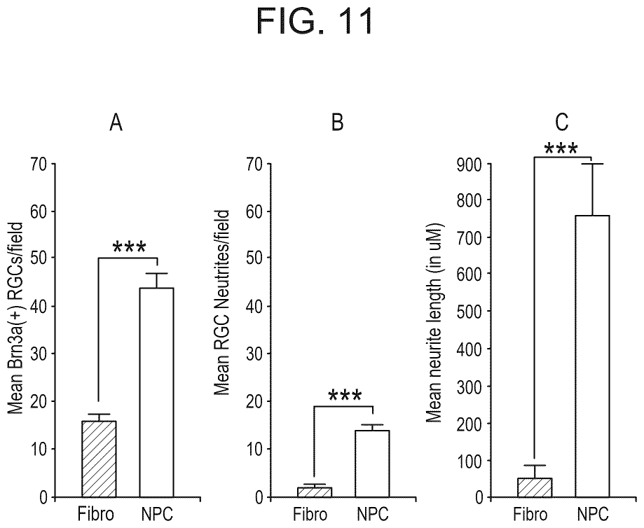

Rat ONH EVs also enhanced RGC neurite extension in culture. Untreated RGC resulted in increased numbers of both Tuj1(+) neurites from Brn3a(+) RGCs in retina cultures. (FIGS. 11A and B).

The response of rat RGCs in primary cultures to EVs derived from cultures of both rat primary fibroblasts and ONLR-NPCs was evaluated. Immediately following primary plating, EVs were administered to the cultures at levels equivalent to twice the original culture medium concentration. Cells were evaluated 3 days post-plating. ONLR-NPC EV-treated retinal cultures yielded nearly 3× as many Brn3a(+) RGCs per field as did fibroblast EV-treated cultures (16±RGCs for fibroblast EVs v. 43.7± for RGCs in cultures treated with ONLR-NPC-EVs). There were nearly 7× as many RGCs expressing neurites in ONLR-NPC EV treated cultures, compared with cultures treated with fibroblast EVs (2± for fibroblast EVs v. 14± for ONLR-NPC-treated EVs). EVs from ONLR-NPCs also stimulated neurite outgrowth, measured in neurite length, to a far greater degree than did EVs from fibroblasts (53.3 um± for fibroblast-EV treated RGCs v. 759.2 um± for ONLR-NPC EV-treated RGCs). Thus, factors associated with ONLR-NPC-EVs both support RGC survival and RGC neurite extension.

Within the group of EV-associated proteins, multiple peptides with neuroprotective properties were identified. These included Gelsolin, Osteopontin, Colony stimulating factor-1 (CSF1), ceruloplasmin, angiotensin, Thrombospondin, PEDF/Serpin F1, Glutathione S-transferase P and IGF1. However, a number of factors associated with neurodegeneration were also identified, including Periostin, SerpinA3, and Somatomedin B (Vitronectin). Since ONH cell cultures include multiple non-NPC cell types (microglia, vascular components, OPCs, oligodendrocyte) as well as NPCs, it is likely that many of these proteins derive from non-NPC exosomes. Additionally the Matrigel medium used to establish and maintain these cultures may contribute contaminants, since cultivation of ONLR-NPCs require high matrigel concentrations (11-15%).

To identify ONLR-NPC-EV specific protein contributions, EV-proteome expression was cross-correlated with ONH sc-seq data. ONLR-NPC EVs exclusively contributed A2M, Cp, and angiotensin, while strongly secreting PEDF/Serpinf1, TF, OSTF1, and Gelsolin, along with a number of other cell types. A similar in silico analysis was performed using the RGC stress response database, evaluating whether post-stress RGCs increase their intracellular requirement for secreted proteins without known receptors. These results are shown in FIG. 10.

### Protocol 8.

As indicated above, the peptide factors disclosed herein were identified by analysis of exosomes secreted by primary optic nerve head cultures. This was performed using isolation of the exosomes (extracellular vesicles) and then performing tandem high pressure liquid chromatography-mass spectroscopy (HPLC-MS/MS) to isolate the individual peptides and identify them via proteomic analysis. The data from the initial HPLC-MS/MS was then correlated against the gene expression data generated by single cell sequencing.

As discussed above, single cell sequencing data was obtained by obtaining cells from dissociated rat optic nerve heads from 20 d old animals. This is followed by individual labeling of the cells using unique DNA identifiers and then generating cDNA from each cell that has the unique DNA identifier, followed by bioinformatic analysis. This yielded over 20,000 individual gene signatures, which were matched to the data from the proteomic analysis.

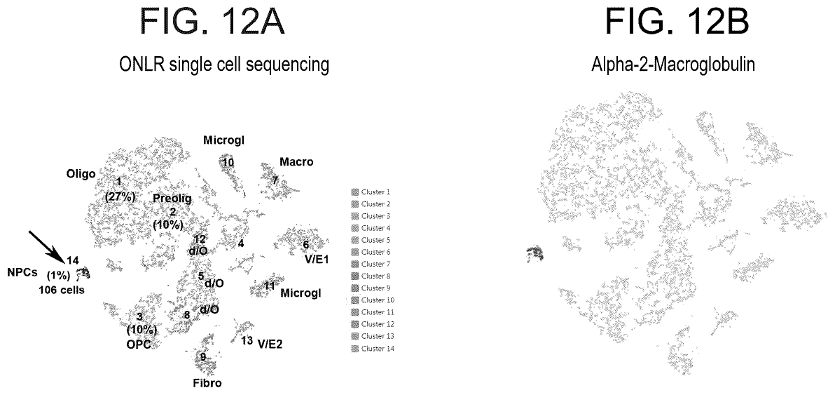

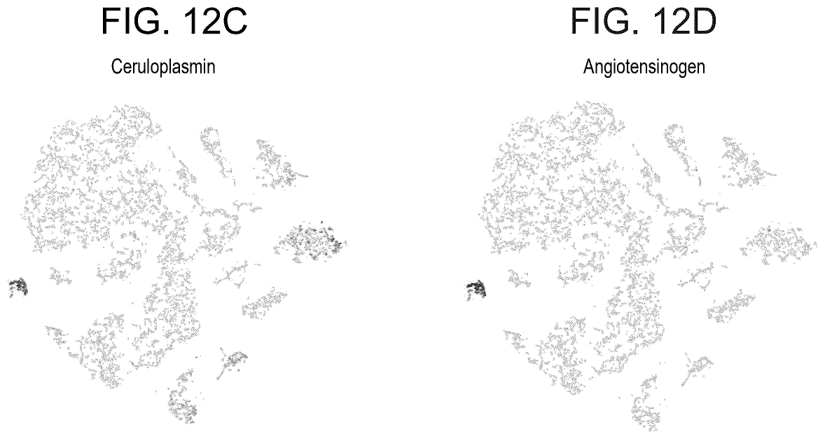

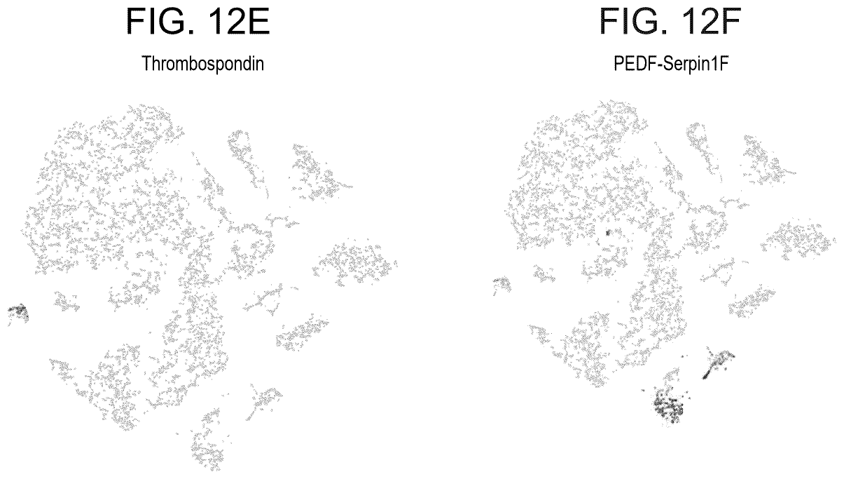

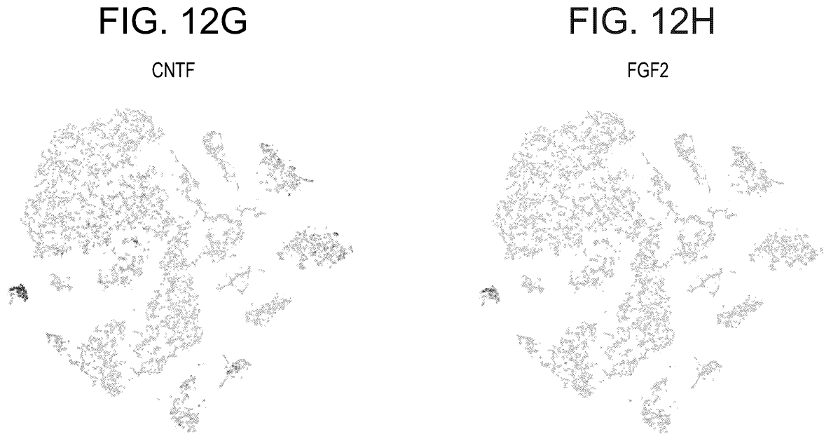

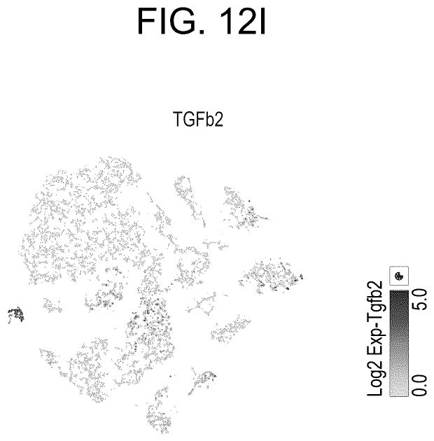

Confirmation of ONLR-NPC expression from ONH single cell analysis is shown in FIG. 12(A-I), where FIG. 12(A) is map of identified cell types from the single cell sequencing and the group of ONLR-NPCs are indicated by an arrow. Only ONLR-NPCs express alpha-2 macroglobulin (FIG. 12(B)), angiotensinogen (FIG. 12(D)) and thrombospondin (FIG. 12(E)). Ceruloplasmin is expressed at highest levels in ONLR-NPCs, but is also expressed at lower levels in fibroblast, vascular endothelium (VE1) and VE2 cell types (FIG. 12(C)). PEDF/Serpin1F (FIG. 12(F)) is expressed in lower levels in ONLR-NPCs, and in higher levels in fibroblasts and VE2 cells. Single cell sequencing shows that the ONLR-NPCs express (and then secrete) CNTF (FIG. 12(G)), FGF2 (FIG. 12(H)) and TGFb2 (FIG. 12(1)) growth factors at high levels.

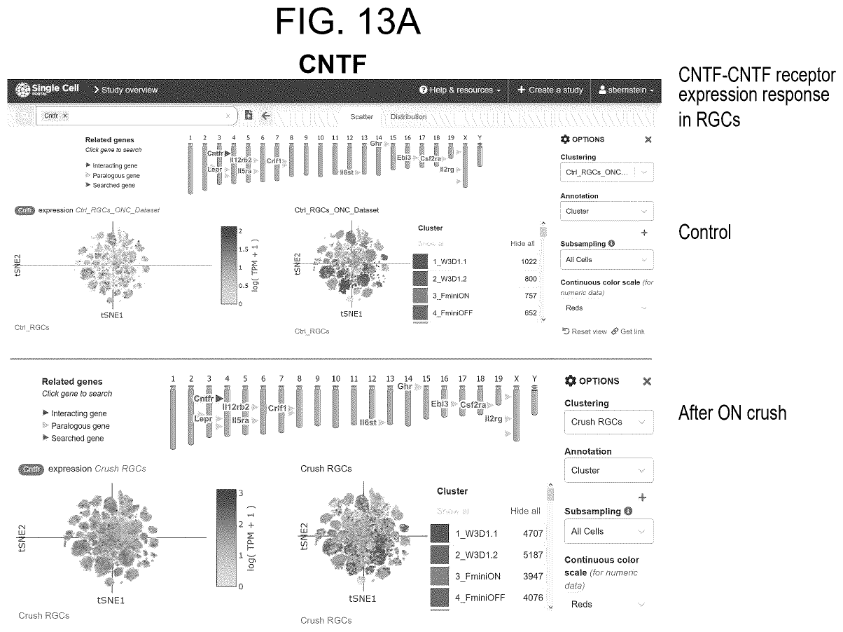

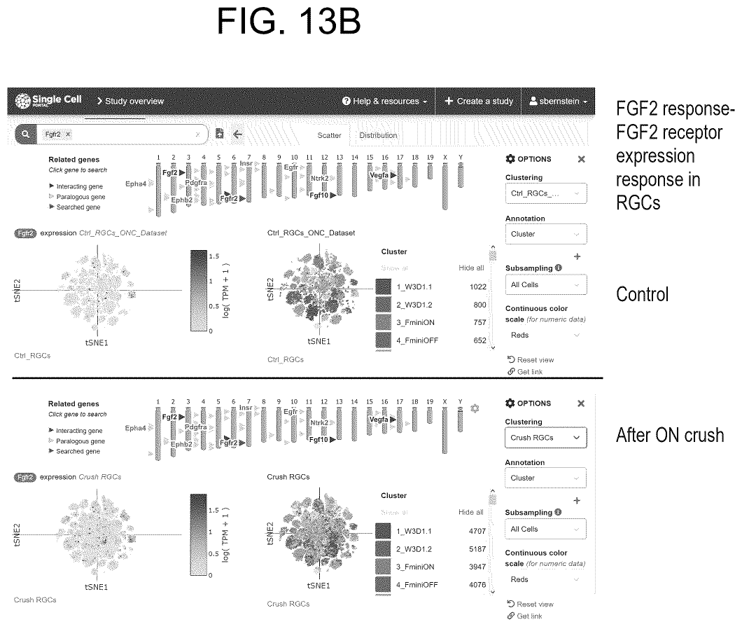

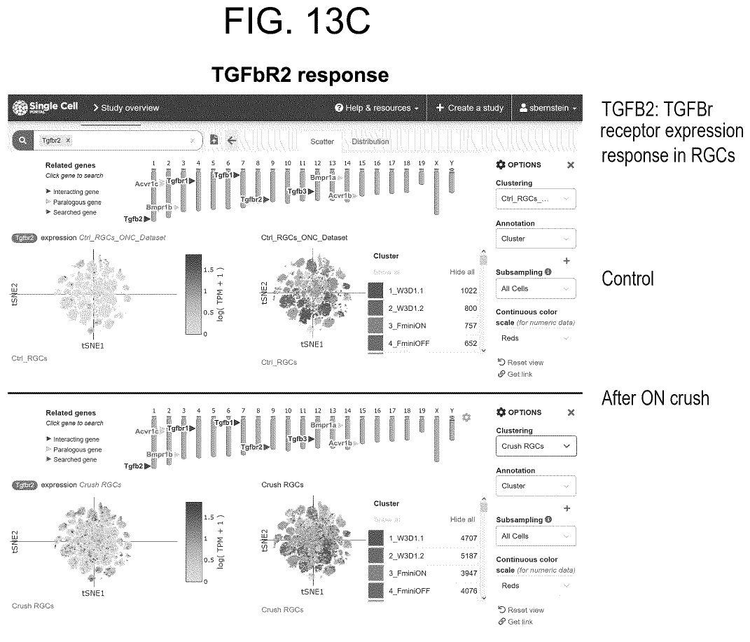

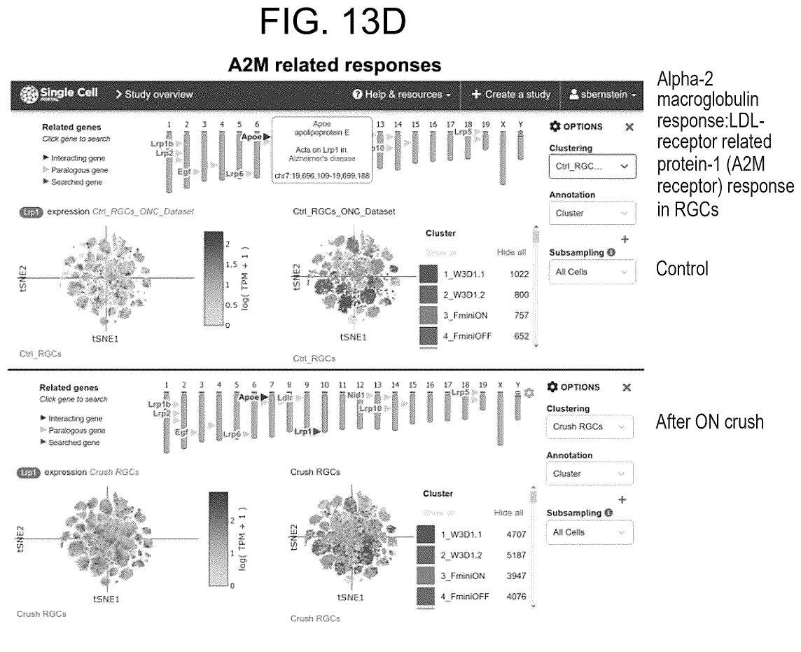

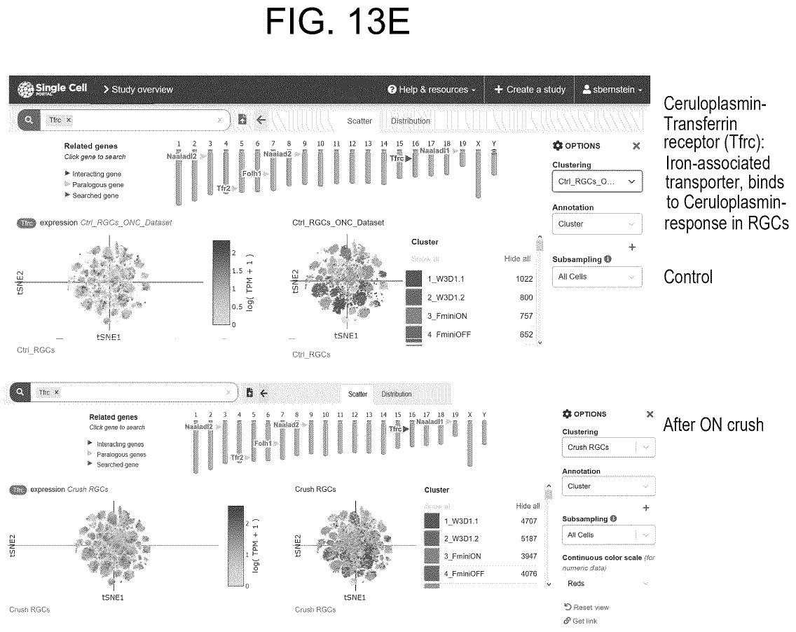

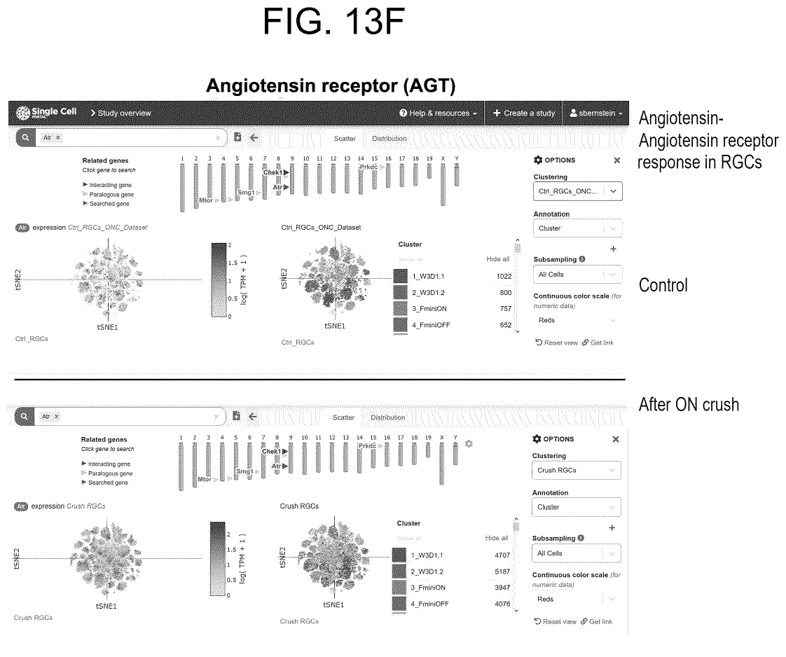

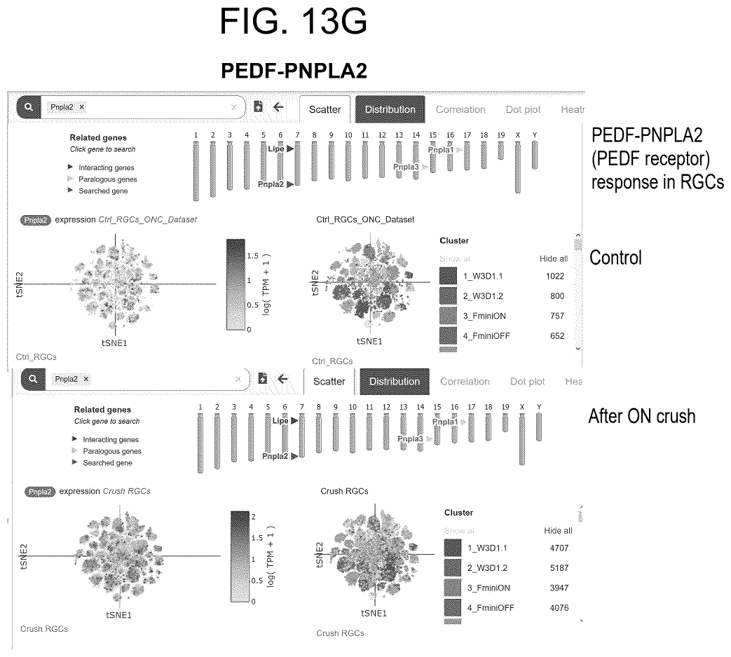

The growth factors identified above were then confirmed to be required for retinal ganglion cell (RGC) function when under stress by evaluating RGC expression of 1) their complementary growth factor receptors (direct secretion) or 2) RGC metabolic associated factors requiring their added function after stress. RGC stress was generated by crushing the optic nerve of a mouse, and analyzing the RGC gene expression from 1-7 days after crush, using single cell sequencing [66]. The results are shown in FIG. 13.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application.
1. Anderson D R, Hoyt W F. Ultrastructure of intraorbital portion of human and monkey optic nerve. Arch Ophthalmol. 1969; 82:506-30.
2. Tesser R A, Niendorf E R, Levin L A. The morphology of an infarct in nonarteritic anterior ischemic optic neuropathy. Ophthalmology. 2003; 110:2031-5.
3. Sun M H, Liao Y J. Structure-Function Analysis of Nonarteritic Anterior Ischemic Optic Neuropathy and Age-Related Differences in Outcome. J Neuroophthalmol. 2017; 37:258-64.
4. Moro F, Doro D, Mantovani E. Anterior ischemic optic neuropathy and aging. Metab Pediatr Syst Ophthalmol. 1989; 124:46-57.
5. Chauhan B C, Mikelberg F S, Balaszi A. Canadian glaucoma study: 2. risk factors for the progression of open-angle glaucoma. Arch Ophthalmol. 2008; 126:1030-6.
6. Calkins D J, Pekny M, Cooper M L, Benowitz L. The challenge of regenerative therapies for the optic nerve in glaucoma. Exp Eye Res. 2017; 157:28-33.
7. Greco A, Rizzo M I, De Virgilio A, Gallo A, Fusconi M, de Vincentiis M. Emerging Concepts in Glaucoma and Review of the Literature. Am J Med. 2016; 129:1000.e7-.e13.
8. Bernstein S L, Guo Y, Kerr C, Fawcett R W, Stern J H, Temple S, et al. The optic nerve lamina region is a neural progenitor cell niche Proc Natl Acad Sci USA. 2020; 117:19287-98
9. Tavazoie, M., Van der Veken, L., Silva-Vargas, V., Louissaint, M., Colonna, L., Zaidi, B., Garcia-Verdugo, J. M., and Doetsch, F. (2008). A specialized vascular niche for adult neural stem cells. Cell Stem Cell 3, 279-288.
10. Albrecht, M. C. (2008). Comparative anatomy of the optic nerve head and inner retina in non-primate animal models used for glaucoma research [In Process Citation]. Open Ophthalmol J 2, 94-101.
11. Goldenberg-Cohen, N., Guo, Y., Margolis, F. L., Miller, N. R., Cohen, Y., and Bernstein, S. L. (2005). Oligodendrocyte Dysfunction Following Induction of Experimental Anterior Optic Nerve Ischemia. In Invest Ophthalmol Vis Sci, pp. 2716-2725.
12. Morrison, J. C., Johnson, E. C., Cepurna, W. O., and Funk, R. H. (1999). Microvasculature of the rat optic nerve head. Invest Ophthalmol Vis Sci 1999 July; 40, 1702-1709.
13. Sun, D., Lye-Barthel, M., Masland, R. H., and Jakobs, T. C. (2009). The morphology and spatial arrangement of astrocytes in the optic nerve head of the mouse. J Comp Neurol 516, 1-9.
14. Davis, C. H., Kim, K. Y., Bushong, E. A., Mills, E. A., Boassa, D., Shih, T., Kinebuchi, M., Phan, S., Zhou, Y., Bihlmeyer, N. A., et al. (2014). Transcellular degradation of axonal mitochondria. Proc Natl Acad Sci USA 111, 9633-9638.
15. Lu, Z., Elliott, M R., Chen, Y., Walsh, J. T., Klibanov, A. L., Ravichandran, K. S., and Kipnis, J. (2011). Phagocytic activity of neuronal progenitors regulates adult neurogenesis. Nat Cell Biol 13, 1076-1083.
16. Ffrench-Constant, C., Miller, R. H., Burne, J. F., and Raff, M. C. (1988). Evidence that migratory oligodendrocyte-type-2 astrocyte (O-2A) progenitor cells are kept out of the rat retina by a barrier at the eye-end of the optic nerve. Journal of Neurocytology 17, 13-25.
17. Fischer, A. J., and Reh, T. A. (2001). Muller glia are a potential source of neural regeneration in the postnatal chicken retina. Nat Neurosci 4, 247-252.
18. Nickerson, P. E. B., Da Silva, N., Myers, T., Stevens, K., and Clarke, D. B. (2008). Neural progenitor potential in cultured Muller glia: Effects of passaging and exogenous growth factor exposure. Brain Research 1230, 1-12.
19. Salero, E., Blenkinsop, T. A., Corneo, B., Harris, A., Rabin, D., Stern, J. H., and Temple, S. (2012). Adult human RPE can be activated into a multipotent stem cell that produces mesenchymal derivatives. Cell Stem Cell 10, 88-95.
20. Davies J E, Huang C, Proschel C, Noble M, Mayer-Proschel M, Davies S J. Astrocytes derived from glial-restricted precursors promote spinal cord repair. J Biol. 2006; 5:7.2-7.21.
21. Gage F H, Temple S. Neural stem cells: generating and regenerating the brain. Neuron. 2013; 80:588-601.
22. Hsu Y-C, Lee D-C, Chiu I-M. Neural Stem Cells, Neural Progenitors, and Neurotrophic Factors. Cell Transplantation. 2007; 16:133-50.
23. Goldman S A, Sim F. Neural progenitor cells of the adult brain. Novartis Found Symp. 2005; 265:66-97.
24. Encinas J M, Sierra A. Neural stem cell deforestation as the main force driving the age-related decline in adult hippocampal neurogenesis. Behav Brain Res. 2012; 227:433-9.
25. Kang S H, Fukaya M, Yang J K, Rothstein J D, Bergles D E. NG2+ CNS Glial Progenitors Remain Committed to the Oligodendrocyte Lineage in Postnatal Life and following Neurodegeneration. Neuron. 2010; 68:668-81.
26. Chen Y, Balasubramaniyan V, Peng J, Hurlock E C, Tallquist M, Li J, et al. Isolation and culture of rat and mouse oligodendrocyte precursor cells. Nature Protocols. 2007; 2:1044-51.
27. Hall P E, Lathia J D, Miller N G, Caldwell M A, Ffrench-Constant C. Integrins are markers of human neural stem cells. Stem Cells. 2006; 24:2078-84.
28. Dimou L, Gotz M. Glial cells as progenitors and stem cells: new roles in the healthy and diseased brain. Physiol Rev. 2014; 94:709-37.
29. Ahlenius H, Visan V, Kokaia M, Lindvall O, Kokaia Z. Neural stem and progenitor cells retain their potential for proliferation and differentiation into functional neurons despite lower number in aged brain. J Neurosci. 2009; 29:4408-19.
30. Tang Y, Wang J, Lin X, Wang L, Shao B, Jin K, et al. Neural stem cell protects aged rat brain from ischemia-reperfusion injury through neurogenesis and angiogenesis. J Cereb Blood Flow Metab. 2014; 34:1138-47.
31. Schultz M B, Sinclair D A. When stem cells grow old: phenotypes and mechanisms of stem cell aging. Development. 2016; 143:3.
32. Lu P, Jones L L, Snyder E Y, Tuszynski M H. Neural stem cells constitutively secrete neurotrophic factors and promote extensive host axonal growth after spinal cord injury. Exp Neurol. 2003; 181:115-29.
33. Morton M C, Neckles V N, Seluzicki C M, Holmberg J C, Feliciano D M. Neonatal Subventricular Zone Neural Stem Cells Release Extracellular Vesicles that Act as a Microglial Morphogen. Cell Rep. 2018; 23:78-89.
34. Kokaia Z, Martino G, Schwartz M, Lindvall O. Cross-talk between neural stem cells and immune cells: the key to better brain repair?Nat Neurosci. 2012; 15:1078-87.
35. Zhang Y, Kim M S, Jia B, Yan J, Zuniga-Hertz J P, Han C, et al. Hypothalamic stem cells control ageing speed partly through exosomal miRNAs. Nature. 2017; 548:52-7.
36. Xin H, Katakowski M, Wang F, Qian J Y, Liu X S, Ali M M, et al. MicroRNA cluster miR-17-92 cluster in exosomes enhance neuroplasticity and functional recovery after stroke in rats. Stroke. 2017; 48:747-53.
37. Yang J, Zhang X, Chen X, Wang L, Yang G. Exosome mediated delivery of miR-124 promotes neurogenesis after ischemia. Mol Ther Nucleic Acids. 2017; 7:278-87.
38. Vogel A, Upadhya R, Shetty A K. Neural stem cell derived extracellular vesicles: Attributes and prospects for treating neurodegenerative disorders. EBioMedicine. 2018; 38:273-82.
39. Zhang Y, Chopp M, Meng Y, Katakowski M, Xin H, Mahmood A, et al. Effect of exosomes derived from multipluripotent mesenchymal stromal cells on functional recovery and neurovascular plasticity in rats after traumatic brain injury. J Neurosurg. 2015; 122:856-67.
40. Mead B, Amaral J, Tomarev S. Mesenchymal stem cell-derived small extracellular vesicles promote neuroprotection in rodent models of glaucoma. Invest Ophthalmol Vis Sci. 2018; 59:702-14.
41. Mead B, Chamling X, Zack D J, Ahmed Z, Tomarev S. TNFα-mediated priming of mesenchymal stem cells enhances their neuroprotective effect on retinal ganglion cells. Invest Ophthalmol Vis Sci. 2020; 61:6 (p1-10).
42. Zhang G, Zhu Z, Wang H, Yu Y, Chen W, Waqas A, et al. Exosomes derived from human neural stem cells stimulated by interferon gamma improve therapeutic ability in ischemic stroke model. Journal of advanced research. 2020; 24:435-45.
43. Zhao, C., Ma, D., Zawadzka, M., Fancy, S. P., Elis-Williams, L., Bouvier, G., Stockley, J. H., de Castro, G. M., Wang, B., Jacobs, S., et al. (2015). Sox2 Sustains Recruitment of Oligodendrocyte Progenitor Cells following CNS Demyelination and Primes Them for Differentiation during Remyelination. J Neurosci 35, 11482-11499.
44. Tang Y-T, Huang Y-Y, Zheng L, Qin S-H, Xu X-P, An T-X, et al. Comparison of isolation methods of exosomes and exosomal RNA from cell culture medium and serum. Int J Mol Med. 2017; 40(3):834-44.
45. Bernstein S L, Guo Y, Kelman S E, Flower R W, Johnson M A. Functional and cellular responses in a novel rodent model of anterior ischemic optic neuropathy. Invest Ophthalmol Vis Sci. 2003; 44:4153-62.
46. Chen C S, Johnson M A, Flower R A, Slater B J, Miller N R, Bernstein S L. A Primate Model of Nonarteritic Anterior Ischemic Optic Neuropathy (pNAION). Invest Ophthalmol Vis Sci. 2008; 49:2985-92.
47. Mehrabian Z, Guo Y, Weinreich D, Bernstein S L. Oligodendrocyte death, neuroinflammation, and the effects of minocycline in a rodent model of nonarteritic anterior ischemic optic neuropathy (rNAION). Molecular Vision. 2017; 23:963-76.
48. Guo Y, Mehrabian Z, Johnson M A, Albers D S, Rich C C, Baumgartner R A, et al. Topical trabodenoson Is neuroprotective in a rodent model of anterior ischemic optic neuropathy (rNAION). Translat Vis Sci Technol. 2019; 8:47-57.
49. Nicholson J D, Puche A C, Guo Y, Weinreich D, Slater B J, Bernstein S L. PGJ2 provides prolonged CNS stroke protection by reducing white matter edema. PLoS One. 2012; 7(12):e50021.
50. Miller N, Johnson M, Nolan T, Guo Y, Bernstein A, Bernstein S. Sustained neuroprotection from a single intravitreal injection of PGJ2 in a non-human primate model of non-arteritic anterior ischemic optic neuropathy. Invest Ophthalmol Vis Sci. 2014; 55:7047-56.
51. Guo Y, Johnson M, Mehrabian Z, Mishra M, Kannan R, Miller N, et al. Dendrimers target the ischemic lesion in rodent and primate models of nonarteritic anterior ischemic optic neuropathy. PLoS One. 2016; 11:e0154437.
52. Yin Y, Cui Q, Gilbert H Y, Yang Y, Yang Z, Berlinicke C, et al. Oncomodulin links inflammation to optic nerve regeneration. Proc Natl Acad Sci USA. 2009; 106:19587-92.
53. Moghimi S, Zangwill L M, Manalastas PIC, Suh M H, Penteado R C, Hou H, Hasenstab K, Ghahari E, Bowd C, Weinreb R N. Association Between Lamina Cribrosa Defects and Progressive Retinal Nerve Fiber Layer Loss in Glaucoma. JAMA Ophthalmology. 2019; 137(4):425-33.
54. Chauhan B C, Mikelberg F S, Balaszi A. Canadian glaucoma study: 2. risk factors for the progression of open-angle glaucoma. Archives of Ophthalmology. 2008; 126(8):1030-6.
55. Dai C, Khaw P T, Yin Z Q, Li D, Raisman G, Li Y. Structural Basis of Glaucoma: The Fortified Astrocytes of the Optic Nerve Head Are the Target of Raised Intraocular Pressure. Glia. 2012; 60:13-28.
56. Lye-Barthel M, Sun D, Jakobs T C. Morphology of Astrocytes in a Glaucomatous Optic NerveOptic Nerve Astrocytes in Glaucoma. Investigative Ophthalmology & Visual Science. 2013; 54(2):909-17.
57. Ffrench-Constant C, Raff M C. Proliferating bipotential glial progenitor cells in adult rat optic nerve. Nature. 1986; Feb. 6-12; 319(6053):499-502.
58. Khakh B S, Sofroniew M V. Diversity of astrocyte functions and phenotypes in neural circuits. Nature Neuroscience. 2015; 18:942.
59. Chen Y, Balasubramaniyan V, Peng J, Hurlock E C, Tallquist M, Li J, Lu Q R. Isolation and culture of rat and mouse oligodendrocyte precursor cells. Nature Protocols. 2007; 2(5):1044-51.
60. Hall P E, Lathia J D, Miller N G, Caldwell M A, Ffrench-Constant C. Integrins are markers of human neural stem cells. Stem Cells. 2006; 24(9):2078-84.
61. Dimou L, Gotz M. Glial cells as progenitors and stem cells: new roles in the healthy and diseased brain. Physiol Rev. 2014; 94(3):709-37.
62. Shen Q, Wang Y, Kokovay E, Lin G, Chuang S M, Goderie S K, Roysam B, Temple S. Adult SVZ stem cells lie in a vascular niche: a quantitative analysis of niche cell-cell interactions. Cell Stem Cell. 2008; 3(3):289-300.
63. Faissner A, Reinhard J. The extracellular matrix compartment of neural stem and glial progenitor cells. Glia. 2015; 63(8):1330-49.
64. Tavazoie M, Van der Veken L, Silva-Vargas V, Louissaint M, Colonna L, Zaidi B, Garcia-Verdugo J M, Doetsch F. A specialized vascular niche for adult neural stem cells. Cell Stem Cell. 2008; 3(3):279-88.
65. Akkermann R, Beyer F, Kury P. Heterogeneous populations of neural stem cells contribute to myelin repair. Neural regeneration research. 2017; 12(4):509-17.
66. Tran M et al. Single-Cell Profiles of Retinal Ganglion Cells Differing in Resilience to Injury Reveal Neuroprotective Genes. Neuron. 2019 104(6):1039-1055.

1. A method of (a) treating or preventing an optic nerve disease, (b) enhancing retinal ganglion cell (RGC) survival, and/or (c) enhancing RGC neurite outgrowth in a subject comprising administering to a subject in need thereof a therapeutically effective amount of a composition comprising one or more of:
(i) optic nerve lamina region neural progenitor cell (ONLR-NPC)-secreted soluble proteins,
(ii) a population of ONLR-NPC-produced extracellular vesicles (EVs),
(iii) ONLR-NPC conditioned media containing ONLR-NPC-secreted soluble proteins and/or ONLR-NPC-produced EVs,
(iv) ONLR-NPC EV lysates, and
(v) ONLR-NPC EV extracts,
thereby (a) treating or preventing an optic nerve disease, (b) enhancing retinal ganglion cell (RGC) survival, and/or (c) enhancing RGC neurite outgrowth in the subject.

2. The method of claim 1, wherein the optic nerve disease is selected from the group consisting of open-angle glaucoma, primary open-angle glaucoma (POAG), angle-closure glaucoma, optic nerve hypoplasia, optic nerve hypomyelination, regional axonal dysfunction, nonarteritic anterior ischemic optic neuropathy (NAION), and optic neuritis.

3. The method of claim 1, wherein the composition is administered to the eye of the subject.

4. The method of claim 1, wherein the composition is administered to the subject via one or more of topical application, subconjunctival injection, intravitreal injection, and retrobulbar injection.

5. The method of claim 1, wherein the composition administered to the subject is a pharmaceutical formulation comprising a pharmaceutically acceptable carrier.

6. The method of claim 1, wherein the subject is a human.

7. The method of claim 1, wherein the ONLR-NPCs are nestin(+), SOX2(+), GFAP(+), HOPX(+), NG2(−) cells.

8. The method of claim 1, wherein the ONLR-NPCs are nestin (+), SOX2(+), GFAP(+), HOPX(+), NG2(−), vimentin(+), BDNF(+) cells.

9. The method of claim 1, wherein the ONLR-NPCs express one or more of Ciliary Neurotrophic Factor (CNTF), Fibroblast Growth Factor 1 (FGF-1), Fibroblast Growth Factor 2 (FGF-2), Insulin-like Growth Factor 2 (IGF-2), Midkine (Neurite Growth-promoting Factor 2), Platelet-derived Growth Factor alpha (PDGFA), Platelet-derived Growth Factor C (PDGFC), Transforming Growth Factor-Beta 1 (TGF-β1), Transforming Growth Factor-Beta 2 (TGF-β2), and Vascular Endothelial Growth Factor A (VEGFA).

10. The method of claim 1, wherein the ONLR-NPCs express at least five growth factors selected from the group consisting of CNTF, FGF-1, FGF-2, IGF-2, Midkine, PDGFA, PDGFC, TGF-β1, TGF-β2, and VEGFA.

11. The method of claim 1, wherein the ONLR-NPCs express each of CNTF, FGF-1, FGF-2, IGF-2, Midkine, PDGFA, PDGFC, TGF-β1, TGF-β2, and VEGFA.

12. The method of claim 1, wherein the ONLR-NPCs express of one or more of the proteins listed in Table 2.

13. The method of claim 1, wherein the ONLR-NPCs express each of the proteins listed in Table 2.

14. The method of claim 1, wherein the composition comprises one or more factors selected from the group consisting of CNTF, FGF-1, FGF-2, IGF-2, Midkine, PDGFA, PDGFC, TGF-β1, TGF-β2, and VEGFA.

15. The method of claim 1, wherein the composition comprises each of CNTF, FGF-2 and TGF-β2.

16. The method of claim 1, wherein the composition comprises each of CNTF, FGF-1, FGF-2, IGF-2, Midkine, PDGFA, PDGFC, TGF-β1, TGF-β2, and VEGFA.

17. The method of claim 1, wherein the EV extract comprises one or more of Alpha-2 macroglobulin, Angiotensinogen, an Angiotensinogen peptide fragment, Angiotensin, Antithrombin 3-Serpin C1, CD9, CD81, Ceruloplasmin, Insulin-like Growth Factor-1 (IGF-1), Leucine rich repeat LGI family member 4 (LGI4), Pigment Epithelium-derived Factor (PEDF), Osteogenic growth factor 1 (OSTF1), Thrombospondin-1, and Adipocyte Enhancing Binding Protein-1 (AEBP1).

18. The method of claim 1, wherein the EV extract comprises each of Alpha-2 macroglobulin, Antithrombin 3-Serpin C1, CD9, CD81, Ceruloplasmin, IGF-1, LGI4, PEDF, OSTF1, Thrombospondin-1, AEBP1, and one or more of Angiotensinogen, an Angiotensinogen peptide fragment, and Angiotensin.

19. The method of claim 1, wherein the ONLR-NPC EV extract is an artificial EV extract comprising each of Fibroblast Growth Factor 2 (FGF-2); Ciliary Neurotrophic Factor (CNTF); Transforming Growth Factor-Beta 2 (TGF-β2); one or more of Angiotensinogen, an Angiotensinogen peptide fragment, and Angiotensin; Alpha-2 macroglobulin; Thrombospondin-1; Ceruloplasmin; and Pigment Epithelium-derived Factor (PEDF).
